SEQUENCE LISTING (5 sequences)

SEQ ID NO: 1 - DNA, 86 bp, synthetic construct
  source 1..86: mol_type = other DNA
tagggaagagaaggacatatgatgctgccgacgattgactagtacatgaccacttgactgtctttgactagtacatgaccacttga

SEQ ID NO: 2 - DNA, 86 bp, synthetic construct
  source 1..86: mol_type = other DNA
tagggaagagaaggacatatgatagggtgagcatccgcataacaatagtgctgtttagttggcttgactagtacatgaccacttga

SEQ ID NO: 3 - DNA, 86 bp, synthetic construct
  source 1..86: mol_type = other DNA
tagggaagagaaggacatatgatctaacttgaataaataccagcaccagactgcccgcgtttcttcactagtacatgaccacttga

SEQ ID NO: 4 - DNA, 86 bp, synthetic construct
  source 1..86: mol_type = other DNA
tagggaagagaaggacatatgatggacgggtatacactagacaacaacaaggaacactctttcttgactagtacatgaccacttga

SEQ ID NO: 5 - DNA, 86 bp, synthetic construct
  source 1..86: mol_type = other DNA
tagggaagagaaggacatatgatgctttccatgtcgttatcctaggggctgttagctaatttcttgactagtacatgaccacttga

(19) United States
(12) Patent Application Publication
(10) Pub. No.: US 2025/0059548 A1
(43) Pub. Date: Feb. 20, 2025

(54) Aptamer Inactivation of Nucleases in Biological Solutions, Reagents, and Kits Used for Sample Collection, Nucleic Acid Testing, Isolation, and Genomic Characterization

(71) Applicants: Luke Thomas DAUM, San Antonio, TX (US); Johna Sere MARLOWE, San Antonio, TX (US)

(72) Inventors: Luke Thomas DAUM, San Antonio, TX (US); Johna Sere MARLOWE, San Antonio, TX (US)

(21) Appl. No.: 18/623,111

(22) Filed: Apr. 1, 2024

Related U.S. Application Data
(60) Provisional application No. 63/456,469, filed on Apr. 1, 2023.

(51) Int. Cl. C12N 15/115; C12N 15/10; C12Q 1/6806

(52) U.S. Cl. CPC C12N 15/115; C12N 15/1003; C12Q 1/6806; C12N 2310/16

(57) ABSTRACT

Systems and methods are described for the use of DNA or RNA aptamers, or a combination of both, to selectively target and deactivate cellular and ubiquitous nucleases (RNases/DNases) in sample collection mediums and genomics kits. These highly specific DNA/RNA aptamers will improve the preservation and stabilization of RNA and DNA polymers obtained from eukaryotic, prokaryotic, and viral sources. The described methods and use involve the utilization of anti-nuclease binding aptamers for two main purposes: 1) deactivating nucleases to enhance the preservation of RNA/DNA in collected samples within a multi-use specimen collection medium, and 2) inclusion in an ‘RNase Removal Kit’. Furthermore, anti-nuclease aptamers can be integrated into existing technologies to minimize nucleic acid degradation and boost RNA/DNA stability in aqueous mixtures, such as chemical/enzyme compositions, buffers, solutions, collection mediums, reagents, media preparations, and nucleic acid testing (NAT) kits used for routine pathogen and disease diagnostics and detection, genomic sequencing and characterization, epigenetic analysis, and nucleic acid purification/extraction.

### CROSS REFERENCES TO RELATED APPLICATIONS

This application claims the benefit under Title 35 United States Code § 119(e) of U.S. Provisional Patent Application Ser. No. 63/456,469; Filed: Apr. 1, 2023; the full disclosure of which is incorporated herein by reference.

### INCORPORATION BY REFERENCE STATEMENT

The contents of the electronic sequence listing (1731009801059.xml); Size: 5,933 bytes; and Date of Creation: Nov. 7, 2024; is herein incorporated by reference in its entirety.

### BACKGROUND OF THE INVENTION

### 1. Field of the Invention

The present invention relates to novel methods and systems for rapidly inactivating nucleases, specifically RNase and DNase, in biological matrices. This invention is particularly beneficial for use in molecular biological kits, specimen collection media, genomic sample preparations, extraction methods, and nucleic acid testing reagents and preparations where the preservation and stabilization of RNA and DNA are crucial for downstream nucleic acid detection and characterization processes. Also disclosed is a formulation and method for an optimized collection medium that gently disrupts biological membranes and subsequently inactivates nucleases by binding aptamers, ensuring safe, non-toxic collection, nuclease inactivation, and preprocessing of clinical, environmental, and biological samples. This embodiment of the invention provides a safe and ambient temperature medium and method for collecting, preserving, and stabilizing RNA/DNA from a collected sample that is unique in the field. In a second embodiment of this invention, anti-nuclease aptamers are immobilized onto paramagnetic beads or affinity resin columns for the effective removal of nucleases in preparations used prior to RNA sequencing, transcriptome analysis, and other genomic applications.

### 2. Description of the Related Art

The field of molecular diagnostics and genomics is rapidly evolving. There is an urgent need to ensure that precious nucleic acids, such as RNA and DNA, especially those processed with molecular biology “wet” reagents found in kits used for sample collection, nucleic acid tests (NAT), RNA/DNA extraction, and genomic applications, are preserved and stabilized from degradation and hydrolysis by cellular released, contaminating, and ‘ubiquitous’ nucleases. Nucleic acids that are processed, purified, amplified, or enriched for detection and genetic characterization in reagent blends or sample collection kits are highly vulnerable to nuclease digestion and degradation, especially over time or at elevated temperatures. Therefore, great care and specialized laboratory handling precautions must be taken, and reagents must be stored at frozen or refrigerated temperatures to ensure stability.

Nucleases pose a particular challenge in molecular biology reagent kits, such as next-generation sequencing (NGS) library preparation kits, quantitative polymerase chain reaction (qPCR) master-mix reagents, RNA/DNA extraction and purification kits, and specimen collection mediums composed of optimized reagent blends, buffers, and washing solutions necessary for the detection, amplification, collection, processing, and purification of small quantities of nucleic acids from biological sources. Nuclease digestion of nucleic acids is further exacerbated when biological specimens or environmental samples, containing high levels of cellular-derived nucleases, are collected at point-of-care, through home-collection, triage sites, remote testing locations, or intermittently from collection sites, pharmacies, or physician's offices where specimens may sit dormant for hours to days at ambient temperatures or higher until processed by molecular diagnostic testing laboratories.

### Nucleases (RNase and DNase

Nucleases are protein enzymes that cleave phosphodiester bonds of nucleic acids. They can be endonucleases or exonucleases, DNases or RNases, topoisomerases, recombinases, ribozymes, or RNA splicing enzymes. Their structures and activities are well characterized, and they belong to several families classified by reaction mechanism, metal ion dependence, and biological function. These functions range from replication, recombination, repair, RNA maturation, processing, interference, defense, nutrient regeneration, to cell death.

RNases are an important family of nucleases found in microbes and cell types from all organisms including prokaryotes and eukaryotes. They are particularly abundant in cells collected from human and animal specimens, bacterial and fungal samples, and environmental samplings including, but not limited to cultures, blood, saliva, sputum, oral fluids, nasopharyngeal, nasal, buccal, urine, and genital swabs and fluids. Since the sugar component of RNA, i.e., ribose contains a hydroxyl group in the 2′ and 3′ position, RNA is chemically much more highly reactive compared to DNA. Therefore, labile RNA is vulnerable to cleavage by RNase with varying specificities that ultimately cleave diester bonds linking phosphate and ribose residues. RNases can be excreted from cells and are also released in high concentrations after cellular lysis. Additionally, RNase is present on the skin. Thus, important care is required to prevent contamination of laboratory bench tops, pipettors, and the reagents used for handling, isolating and characterizing nucleic acids. The problem is compounded because no solution exists for readily inactivating RNases from reagents, media, and buffers. Because of the presence of intrachain disulfide bonds, RNase is resistant to elevated temperature and mild denaturants and can refold quickly when denatured. Furthermore, RNases (unlike DNases) do not require divalent cations for activity and thus cannot be easily inactivated by the inclusion of chelators, e.g., ethylenediaminetetraacetic acid (EDTA), sodium citrate, etc. in buffer solutions. Because nucleases like RNase are abundantly present in most biological samples collected, they present a persistent problem in the laboratory, especially diagnostic, forensic, or genomics laboratories that are detecting, amplifying, manipulating and characterizing minute amounts of RNA/DNA from clinical samples, cell cultures, or microbes.

### NGS, Long Read Sequencing, RNASeq, and Transcriptomics Sequencing Kits and Reagents

Library preparation is the first step of next generation sequencing (NGS). It allows DNA or cDNA produced from RNA template to adhere to the sequencing flow cell and enables the individual nucleotides (A, T, C, and G's) from DNA to be sequenced using ligation-based or tagmentation-based NGS approaches. NGS manufacturers for library preparation kits, such as Illumina (San Diego, CA, USA), ThermoFisher (Waltham, MA, USA), Oxford Nanopore Technologies (Oxford, UK), Pacific BioSciences (Menlo Park, CA, USA), and Element BioSciences (San Diego, CA, USA) produce a variety of commercially available kits for use on their sequencing platforms. Several other companies such as New England BioLabs Inc. (Ipswich, MA, USA), 10× Genomics (Pleasanton, CA, USA), JumpCode (Carlsbad, CA, USA), etc. market and produce commercial genomics kits for library preparation, subtractive depletion, RNA transcription, and epigenic analysis for genomic and metagenomic applications that are ‘platform agnostic’ and can be used upstream and prefatory for subtracting unwanted genomic materials and manipulating, cleaning, purifying and preparing nucleic acids for NGS or traditional genomic characterization. RNA-seq (RNA-sequencing) is a technique for characterizing and quantifying RNA sequences in a sample through NGS analysis. The RNA-seq method uses RNA for characterizing the transcriptome, indicating which genes encoded in DNA are actively being expressed. The recommended input for most library kits is typically 0.5-200 ng of DNA or cDNA, and there are several steps that employ reagents, buffers, solutions, and bead-based binding and washing steps for processing and preparing RNA genomic libraries that originate from ‘crude’ biological specimens that are highly vulnerable to degradation by nuclease digestion.

Long-read sequencing is a powerful technology that allows for the sequencing of longer DNA fragments, enabling the detection of complex genomic structures and variations. Two leading platforms in long-read sequencing are Pacific Biosciences and Oxford Nanopore. Pacific Biosciences utilizes a single-molecule, real-time sequencing approach, while Oxford Nanopore employs nanopore technology to sequence DNA molecules as they pass through a protein nanopore. These platforms offer new sequencing capabilities, such as the ability to detect structural variations, long-range phasing, and epigenetic modifications, which are not easily achievable with short-read sequencing technologies. By providing longer reads, these platforms are revolutionizing genomic research and opening new possibilities for understanding the complexity of the genome.

While RNA-Seq enables detailed analysis of RNA from a cell or tissue, single cell sequencing takes this a step further by analyzing RNA at the individual cell level, providing insights into cellular heterogeneity. RNA sequencing is crucial for understanding biological processes, disease mechanisms, and identifying potential drug targets. However, obtaining and ensuring the stability of high-quality RNA from collected samples is essential for accurate results, as RNA characterization using next-generation sequencing is expensive. Thus, it is critical to invest in sample collection systems that ensure the integrity of RNA samples to maximize the value of RNA-Seq and single cell sequencing data methods.

### Molecular ‘Master-Mix’ Kits and Reagents for Disease and Biomarker Detection

Several diagnostic detection kits based on nucleic acid testing (NAT) are commercially available for disease detection. These kits typically use optimized reagent blends consisting of salts, buffers, and enzyme blends for traditional polymerase chain reaction (PCR), quantitative-PCR (qPCR), reverse-transcription qPCR (RT-qPCR), loop-mediated isothermal amplification, and non-isothermal amplification methods to detect infectious targets (bacterial, viral, fungal), disease markers, resistance mutations, or human genomic targets.

For example, the TaqPath 1-step RT qPCR MM kit (Thermo Fisher Scientific, Waltham, MA, USA) is one such kit used for RT-qPCR detection of viral infectious disease targets. An example of a non-isothermal method is LumiraDx's SARS-COV-2 RNA STAR Complete kit (LumiraDx, London, England, UK). This kit is an FDA Emergency Use Authorization (EUA) approved molecular test for rapid, qualitative detection of nucleic acid from SARS-COV-2 from respiratory swabs collected from individuals suspected of harboring COVID-19, for contact tracing, or for self-collection formats.

The assay is approved for use with several qPCR platforms, including ThermoFisher's QuantStudio May 7, 2012 (Waltham, MA, USA) instruments, and is unique compared to other molecular tests because it does not require pre-processing steps or tedious nucleic acid extraction. Therefore, the reagents and buffers included in all these kits are subject to degradation by nuclease digestion.

### DNA and RNA Extraction and Purification Kits

Nucleic acid extraction and purification are critical steps in molecular-based nucleic acid detection and are used before most molecular diagnostic kits. It is often used in high-throughput bead-based approaches in large diagnostic laboratories and is also important in point-of-care diagnostics (POC-Dx). In standard nucleic acid extraction, silica spin-columns or para-magnetized silica-coated beads are used to bind and purify RNA and DNA from cellular debris. Most commercial nucleic acid extraction kits use a basic approach that entails four overarching steps to purify nucleic acids: (i) cell disruption (lysis), which involves shearing the cellular lipid bilayer, (ii) binding to silica dioxide beads/columns, (iii) washing to remove unwanted debris, and (iv) nucleic acid elution. Additionally, purification of Poly(A)+ RNA by Oligo(dT)-Cellulose Chromatography, alkaline extraction of plasmid DNA, and Chelex® are other known methods for isolating specific forms of nucleic acids from specialized cell types. Lastly, nucleic acid extraction using forensic applications, and cetyltrimethylammonium bromide (CTAB) extraction of plant nucleic acids are also widely utilized approaches available in commercial kits from Qiagen (Germantown, MD, USA), Zymo (Irvine, CA, USA), and Thermo Fisher Scientific (Waltham, MA, USA).

In recent years, several semi-extraction methodologies, such as mild lysis, pre-processing methods, and protocols, have been developed. These procedures involve disrupting viral, bacterial, or eukaryotic cellular membranes through heating, proteinase K, physical shearing/agitation, sonication, mild chemical disruption, or combinations of these methods. In this process, a collected primary specimen undergoes mild lysis and pretreatment before being added directly to detection blends, such as Master-Mix for molecular detection, without purification or removal of cellular debris (lipids, carbohydrates, proteins) using silica dioxide-based methods. In all cases, the extraction of precious nucleic acid, whether through routine extraction or pre-processing, involves cellular lysis and the release of highly labile RNA/DNA. Once lysed, RNA/DNA polymers are highly susceptible to hydrolysis and fragmentation through nuclease-mediated degradation. To date, no extraction kits include methods or approaches that utilize targeting aptamers to inactivate or remove damaging nucleases from the preparation before nucleic acid purification.

### Primary Specimen Collection Devices and Kits

The first and most critical step in disease detection and genomic analysis is collecting an optimal clinical specimen. This is particularly important for viral RNA diseases such as influenza A/B, respiratory syncytial virus (RSV), adenoviruses, parainfluenza, and severe acute respiratory syndrome coronavirus 2 (SARS-COV-2). Collection of primary biological specimens using sterile swabs is a common approach for procuring oral, nasal/nasopharyngeal, buccal, penile/vaginal samples. Typically, this is achieved by breaking off the swab head containing the collected specimen into a collection vial/tube with media, and then transporting/shipping the vial to a diagnostic laboratory for testing, typically using qPCR or other molecular method. Additionally, collecting urine, sputum, nasal washes, or other bodily fluids necessitates transfer of a fixed fluid volume into tubes/vials containing collection medium intended to preserve and stabilize the specimen until they are processed at the laboratory. There are three families of collection media used widely for primary specimen collection, each with advantages and disadvantages: (a) viral/universal transport medium (VTM/UTM), (b) molecular transport mediums (MTM), and (c) sterile saline or phosphate buffered saline (PBS) solution.

### Viral/Universal Transport Medium (VTM/UTM)

Commercial VTM and UTM are marketed and sold by Copan Diagnostics (Brescia, Italy), Becton-Dickenson (Franklin Lakes, NY, USA), REMEL (Lenexa, KS, USA), and Puritan Medical Products (Guilford, ME, USA). These reagent blends were originally developed for culturing microbes, so their composition is designed to keep microbes alive until they reach the laboratory. This is achieved through osmoregulation of microbial lipid layers. The chemical compositions of VTM/UTM are optimized to preserve and stabilize the integrity of microbial lipid bilayers during shipment and transport to the laboratory. VTMs and UTMs used for transporting viruses, chlamydia, ureaplasmas, and mycoplasmas consist of buffered salts, antibiotics/fungicides to inhibit bacterial and fungal growth, and a pH indicator that gives them their characteristic pink hue. Surprisingly, the widespread use of VTM and UTM for downstream nucleic acid testing has become a common practice in nucleic acid detection and surveillance. Despite the goal of maintaining microbial viability and preservation, VTM/UTM do not lyse cells or preserve and stabilize released nucleic acids. Additionally, specimens collected in VTM and UTM may be potentially infectious, presenting challenges during shipping and transport. Moreover, they contain reagents (e.g., CaCl₂), gelatin, etc.) that can interfere and carry over during nucleic acid extraction, inhibiting qPCR and other molecular detection methods.

### Molecular Transport Mediums (MTM)

Commercial MTM is marketed and distributed by Spectrum Solutions (Draper, UT, USA), Copan Diagnostics (Brescia, Italy), OraSure Technologies (Bethlehem, PA, USA), Longhorn Vaccines & Diagnostics (Bethesda, MD, USA), and Medical Wire (Corsham, Wiltshire, UK). These media are composed of similar reagent blends containing chaotropic salts such as corrosive guanidine thiocyanate/isothiocyanate, and harmful denaturing/surfactants such as sodium dodecyl sulfate (SDS), etc. Some MTMs are even supplemented with alcohol(s) such as ethanol. MTMs are designed to kill microbes by lysing phospholipid bilayers and inactivating nucleases. For specimens collected in MTM, the mechanism of nuclease inactivation is via chemical denaturation of proteins through the action of chaotropic salts and denaturants. Most MTMs consist of reagent profiles commonly found in lysis buffers used in RNA/DNA extraction kits. However, the chemical profiles of MTMs consist of toxic and corrosive chemical compositions that are: i) harmful if accidentally spilled, contacted, or consumed by humans, ii) dangerous to the environment and municipal water treatment facilities, iii) dependent on specialized packaging/vialing to prevent leakage, iv) hazardous to humans and cannot and should not be used at point-of-care, for self-collection, or as direct-to-consumer home collection kits, v) potentially flammable or have low flash-point temperatures, vi) corrosive and reactive with metals, vii) reactive to bleach during cleanup to form potentially toxic cyanide gases.

### Normal Saline (NS) or Phosphate Buffered Saline (PBS) Solutions

Normal saline (NS) or phosphate buffered saline (PBS) solutions are available from multiple vendors for specimen collection. During the COVID-19 pandemic, saline and PBS were commonly used as an alternative to VTM/UTM for detecting SARS-COV-2 by RT-qPCR because NS/PBS are inexpensive and readily accessible. However, a limitation of using samples collected in NS and PBS is that the specimens are vulnerable to immediate nuclease degradation, especially at ambient temperatures or higher. Additionally, cells can be potentially hazardous since there are no chemical additives to aid in lipid bilayer lysis and disruption, and the higher salt content of these mixtures may lead to interference downstream when directly added to PCR ‘master mix’, or other NAT collection, pre-processing, extraction, or testing buffers and reagents.

### The Use of Aptamers

Aptamers are single-stranded DNA or RNA (ssDNA or ssRNA) molecules that can selectively bind to a specific target. The method of engineering aptamers was described over 20 years ago. They are relatively short oligonucleotides that typically range in size from 20 to 100 nucleotides. Like antibodies, aptamers can bind to a variety of molecules including proteins, peptides, carbohydrates, small molecules, toxins, and whole cells. The primary sequence of aptamers forms base pairs with homologous nucleotides, enabling the formation of various helices, loops, and other unique tertiary structures. Aptamers bind through affinity target recognition using a three-dimensional ‘lock-and-key’ attachment, as well as hydrophobic interactions, base-stacking, and intercalation. In many ways, aptamers are nucleotide analogues of antibodies. However, unlike most antibodies, aptamers are neither immunogenic nor toxic. Furthermore, aptamers offer a variety of advantages over antibodies, including low cost of chemical synthesis, thermostability, and consistent performance. Additionally, unlike antibodies, aptamers are ‘benign’ oligos of nucleic acid that do not interfere in molecular methodologies, sample collection, and nucleic acid testing. These important features make aptamers a novel approach and ideal for use in a novel collection kit for safe and eco-friendly specimen collection and a diagnostic kit designed for RNase/DNase removal.

Historically, the widespread use of aptamers in human therapeutic approaches has faced several challenges, including degradation or excretion from the blood, renal filtration, and pharmacokinetic duration/action. Many of these therapeutic challenges have been overcome or partially alleviated through the use of modified nucleotides, end-capping, sugar modifications, and specialized linkages on aptamers. The use of aptamers in diagnostic disease detection has been more successful, with aptamers being used in place of antibodies in western blotting, enzyme-linked immunosorbent assays (ELISA) tests, and biosensors. Furthermore, aptamers applied for diagnostic use and disease detection have fewer limitations related to health risks.

Prior art for sample collection media, such as PBS or saline solution, involves the use of sterile techniques using autoclaving to inactivate ubiquitous nucleases from aqueous solutions. However, this approach does not prevent the introduction of nuclease contamination when collection vials are opened in the field or when nucleases are released and introduced by microbes and cells during routine collection of clinical or environmental samples. The chemical reagent formulation found in MTM will inactivate nucleases but is toxic, harsh to the environment, and potentially lethal when reacted with bleach compounds. Furthermore, when samples are collected in MTM, the RNA/DNA from the sample must first be extracted and purified involving additional cost and steps to remove the chemicals prior to nucleic acid detection. Additionally, since MTM denatures all proteins by chemical disruption, the collected sample cannot be used for any assay that involves protein or antigen testing, such as lateral flow tests, rapid antigen tests, or colorimetric protein-based tests. Thus, MTM is not suited for multiple test types (e.g., lateral flow and rapid antigen tests), or for self-collection, home collection, or safe shipping. To date, the use of aptamers in collection media for targeting, removing, and inactivating nucleases for the purpose of stabilizing and preserving RNA and DNA from collected specimens is novel and has not been previously envisioned or performed. A novel formulation for a collection medium containing: a) aptamers that inactivate nucleases, b) one or more mild detergents for gentle cellular/viral lysis that do not contain protein denaturants/caustic chemicals, c) reagents that function synergistically with PCR ‘master mixes’ and molecular detection mixtures (e.g., isothermal amplification mixes, etc.) without the need to perform prefatory RNA/DNA extraction (i.e., extraction-less), and d) chemical compositions that do not disrupt or denature proteins or antigen targets in lateral flow and rapid antigen testing is currently unavailable. Furthermore, the general use of aptamers that target and inactivate nucleases in nucleic acid-based extraction kits, molecular biology reagents and buffers, or for use in a novel kit that includes immobilized aptamers attached to beads or resin columns for direct nuclease removal is unknown in the art.

### SUMMARY OF THE INVENTION

The systems and methods described here involve an innovative approach that includes the use of one or more RNA or DNA aptamers to deactivate or eliminate problematic nucleases in various types of samples. These samples can be from clinical, veterinary, or environmental sources, as well as biological solutions, reagents, and kits used for collecting, purifying, or detecting/characterizing RNA and DNA samples. Aptamers are particularly advantageous because they do not require refrigeration or freezing to remain active over extended periods. Their small size and unique nucleic acid secondary structure make them ideal for collecting and extracting nucleic acids. This is because they can bind more effectively to silica dioxide-based resins, filters, and magnetized beads during extraction procedures, especially when carrier RNA/DNA is used.

Samples collected in a collection medium or processed with nucleic acid purification and extraction kits that utilize RNA/DNA binding steps with silica-based spin columns or paramagnetic beads are susceptible to degradation by nucleases. By incorporating anti-nuclease aptamers in conjunction with a well-balanced and synergistic profile of reagents for preservation and stability of RNA/DNA, such as a new collection medium substantially different from prior art including VTM/UTM and MTM media; or inclusion into buffers and reagent solutions used for genomic analysis and characterization; or by directly immobilizing them on paramagnetic beads, selection matrices, or silica filters, the preservation and stabilization of RNA and DNA from collected samples or in genomic analysis kits can be significantly improved.

Techniques for RNA analysis, such as reverse transcription polymerase chain reaction (RT-PCR) or next-generation sequencing, can also be used in conjunction with these methods to further enhance the analysis of nucleic acids in various samples. Creating high affinity binding aptamers is an arduous process. Aptamers as oligonucleotides have two sides, a hydrophilic side composed of the phosphate backbone and a hydrophobic side composed of the nucleotides. Aptamer selection is traditionally performed by parsing those aptamers that bind strongly to a target from those that do not in a process termed systematic evolution of ligands by Exponential Enrichment (SELEX). Using SELEX or other derivative of the SELEX process, an aptamer library (consisting of a “selection library” containing millions of random nucleic acid sequences) are applied to immobilized nucleases (the target) and those that bind with high affinity are retained. If the cells do not naturally adhere to a surface, the bound aptamers are selected for by centrifugation of cells and collection of an aptamer “pellet”. This process can be repeated with subsequent washes to increase the stringency of the binding reaction. For binding to RNase, high affinity DNA aptamers are ideal; conversely, for binding to DNase, RNA aptamers are generated. There are several reports on the discovery and use of aptamers for pathogen detection and human therapeutic use. However, none of these discoveries have utilized aptamers for direct target and inactivation of nuclease enzymes.

This invention focuses on the fabrication and use of aptamers for targeting and inactivating nucleases. Some embodiments of the invention will involve, but are not limited to, the use of highly specific anti-nuclease aptamers for: (a) specimen collection media, (b) removing nucleases for genomic analysis and processing kits, and (c) immobilizing on paramagnetic beads or resin columns for “subtractive” nuclease removal in a novel RNase/DNase deletion kit. Additionally, the invention provides a method for shielding and protecting susceptible RNA and DNA polymers to obtain a population of polynucleotides from a biological sample containing cells and viruses with nucleic acids.

Including aptamers in the sample collection medium to inactivate nucleases generally involves steps such as contacting the specimen (e.g., swab, cells, blood, feces, urine, etc.) with an amount of aqueous media containing anti-nuclease aptamer effective to: (a) lyse a portion of cells to release RNAs and/or DNAs from the sample, and (b) bind and inhibit nucleases in the sample from hydrolyzing or enzymatically degrading RNA and DNA, thereby preserving the population of polynucleotides in the sample. Nucleases, in general, are highly robust enzymes that are resistant to degradation by high heat, salt concentration, and pH. The use of aptamers to bind with high affinity and render a measurable amount of nuclease inactive is a novel approach for preserving and stabilizing quality nucleic acids from samples for subsequent nucleic acid testing and characterization.

In another embodiment, anti-nuclease aptamers are immobilized and optimized for use in a “Nuclease Removal Kit” to inactivate and subtract nucleases from a pool of polynucleotides used before detecting and characterizing RNA and DNA from complex mixtures or samples.

### BRIEF DESCRIPTION OF THE DRAWINGS

FIG. 1 is a composite image of the results of an experiment showing the presence of nucleases (RNases) present in cells or partially lysed cells from a routine human clinical nasopharyngeal (NP) swab collected in a formulation for a novel collection medium, Xtract-Free™ medium, (see below) without any aptamer protection. In the experiment, nucleases released from cells collected from NP swab and placed into the media are observed to enzymatically degrade RNA from the sample.

FIG. 2 is a graph showing a quantitative RT-PCR (qRT-PCR) analysis of single stranded RNA (ssRNA) recovered after incubation in RNase (ribonuclease A) for 2 hours @ 37° C.

FIG. 3 is a schematic overview of a method for using an RNase Removal Kit where high affinity aptamers are covalently attached and immobilized by either 3′ prime or 5′ prime attachment to carboxy coated paramagnetic beads.

FIG. 4 is a method flowchart of the example method shown in FIG. 3 for using an RNase Removal Kit where high affinity aptamers are covalently attached and immobilized by attachment to carboxy coated paramagnetic beads.

FIG. 5 is a method flowchart of a broad method for using a sample/specimen media containing inactivating nuclease aptamers to prevent degradation by nucleases and to stabilize a collected sample for transport and later use in nucleic acid testing.

FIG. 6 is a schematic overview of a method for selecting anti-RNase aptamers from a pool of 10¹⁵ possible candidate sequences. In contrast to typical aptamer library selection, this approach generated high-binding, anti-nuclease inactivation aptamers (showing 8,800-fold to 10,200-fold inactivation compared to control) that were highly enriched and selected for optimal activity a novel sample collection mixture and at elevated ambient temperature (25-30° C.).

FIG. 7 is a graph showing an assessment of enzymatic activity by RNase A enzyme that was chemically immobilized (covalently bonded) to carboxy paramagnetic beads as assessed by qRT-PCR. Three concentrations (10⁶, 10⁵, and 10⁴ genomic copies/mL) of SARS-COV-2 viral RNA were used as a substrate to assess bead-immobilized enzymatic degradation by RNase A enzyme.

FIG. 8 is a graph showing a quantitative time course (0 to 15 minutes) tracking the inactivation of RNase enzyme by select anti-RNase aptamers compared to controls and other nucleic acid species as assessed by fluorometer detection using the RNaseAlert Substrate Nuclease Detection System, following the manufacturer's guidelines (Integrated DNA Technology, Coralville, IA).

FIG. 9 is a graph illustrating the effectiveness of specific anti-RNase aptamers to bind and inactivate the RNase enzyme as determined by qRT-PCR. In comparison to a random 146-nucleotide ssDNA, Poly-A RNA (commonly used as a carrier species in commercial extraction kits), and random hexamers (N6) ssDNA, the aptamers discussed here showed a difference of 14.6 to 16.9 qRT-PCR cycle threshold (C_{T}) values compared to negative control reactions. These C_{T} values equate to 4.4 to 5.1 Log (base 10) differences in RNase inactivation by anti-RNase aptamers, providing 8,800-fold to 10,200-fold protection compared to controls. Average triplicate reactions with standard deviation are shown.

FIG. 10A is a graph and FIG. 10B is a table showing the use of the novel collection media described herein (Xtract-Free) containing anti-RNase aptamers in comparison commercial UTM-RT (Copan Diagnostics, Brescia, Italy) using a commercial molecular detection test (LumiraDx). Shown is a limit of detection for SARS-COV-2 virus in Xtract-Free and Copan UTM-RT medium according to RNA Star Complete and RT-qPCR. FIG. 10A shows RT-qPCR limit of detection by medium type. An average of five replicates for each dilution with standard deviation bars are shown. FIG. 10B shows the limit of detection comparing RNA Star Complete and RT-qPCR in each medium.

FIG. 11 is a table showing an evaluation of 108 human clinical samples collected in Xtract-Free, the novel collection medium described herein. The trial evaluated sensitivity for detection of SARS-COV-2 viral RNA from clinical swabs (N=108) collected in Xtract-Free using the LumiraDx RNA Star Complete as compared to results by standard RT-qPCR. The evaluation with https://www.medcalc.org/calc/diagnostic_test.php.

FIG. 12 is a table showing a comparison of 10 Clinical Nasal Specimens Collected in Xtract-Free and MTM demonstrating the utility of using the same collected sample in Xtract-Free for performing multiple testing type (molecular and rapid antigen) and performing testing with and without nucleic acid extraction.

FIG. 13 is a composite image showing a fluorogenic RNase detection assay in which fluorescence under ultraviolet light indicates that control RNA is actively being degraded by RNases. In this test, RNA degradation by RNase occurs when control RNA is cleaved, releasing a fluorescent marker at the 5′ end from proximity to a fluorescent quencher at the 3′ end of ssRNA. A negative control, where no fluorescence is observed under UV, indicates that RNA is not being degraded due to inactivation by binding aptamers to RNase and that the RNA is fully intact. In this experiment, 4 tubes containing different anti-RNase aptamers were spiked with a molar excess of RNase A enzyme but did not illuminate compared to controls, even after 5 days of incubation.

### DETAILED DESCRIPTION OF THE EXEMPLARY EMBODIMENTS

The present invention provides a method for using a DNA or RNA aptamer or combinations of DNA/RNA aptamers for selective targeting and inactivation of cellular and ubiquitous nucleases (RNases/DNases). In this invention, highly specific DNA/RNA aptamers will enhance preservation and stabilization of RNA and DNA polymers derived from eukaryotic, prokaryotic, and viral sources. Enhanced RNA/DNA preservation by aptamers will be achieved through inactivation, i.e., ‘knockout’ of enzymatic nuclease digestion from samples collected in specimen collection media, aqueous mixtures (e.g., chemical/enzyme compositions, buffers, solutions, reagents, media preparations), and nucleic acid testing (NAT) kits that are used for routine pathogen and disease diagnostics and detection, genomic sequencing and characterization, epigenetic analysis, and nucleic acid purification/extraction.

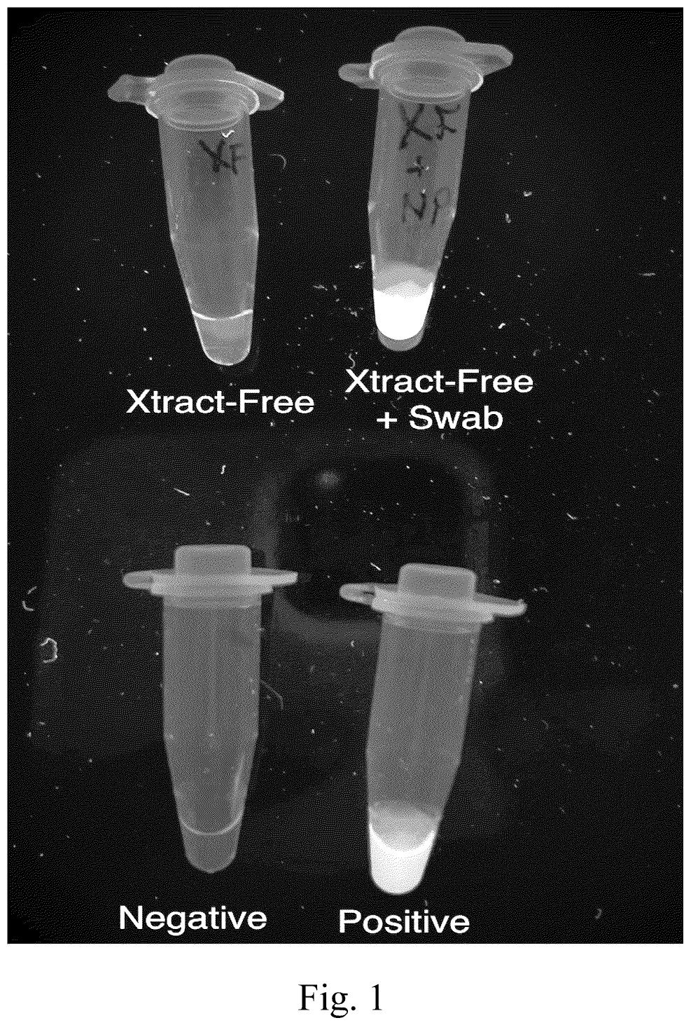

FIG. 1 is a composite image of an experiment showing the presence of nucleases (RNases) present in cells or partially lysed cells from a routine human clinical nasopharyngeal (NP) swab collected in a novel collection medium, Xtract-Free, without aptamer protection. RNase was detected using fluorescence-quenched oligonucleotide probes upon nuclease degradation of RNA (RNaseAlert® (Integrated DNA Technologies (IDT), Coralville, IA, USA). Collection medium with collected NP swab was incubated with detection reagent for 10 minutes. High levels of RNase are prevalent in collected clinical samples degrading precious RNA immediately upon collection. Positive (lower right) and negative (lower left) control reactions included.

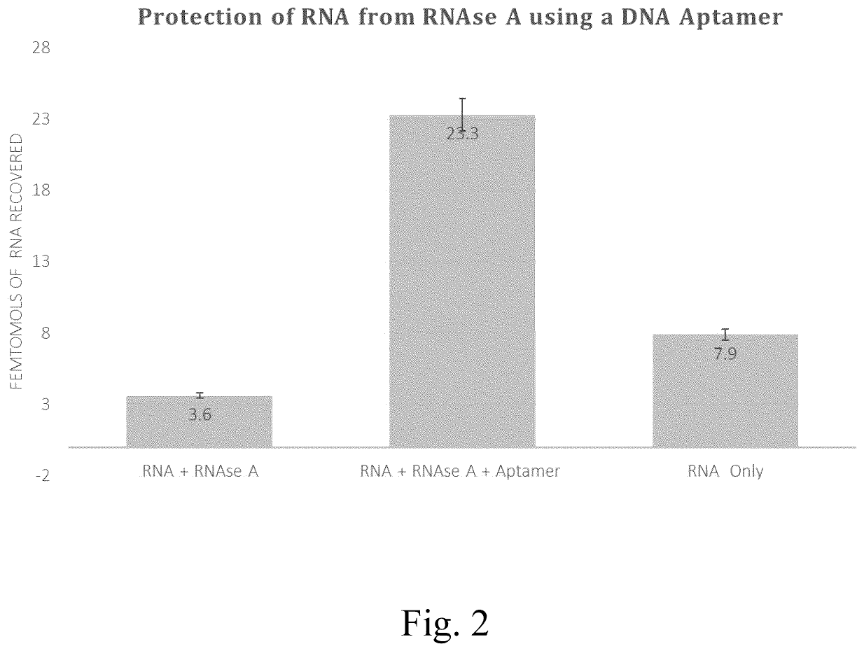

FIG. 2 is a graph showing a quantitative RT-PCR (qRT-PCR) analysis of single stranded RNA (ssRNA) recovered after incubation in RNase (ribonuclease A) for 2 hours @ 37° C. Approximately 5× more ssRNA was recovered in samples containing anti-RNase aptamer compared to equivalent amounts of ssRNA without aptamer protection. Average triplicate reactions are shown.

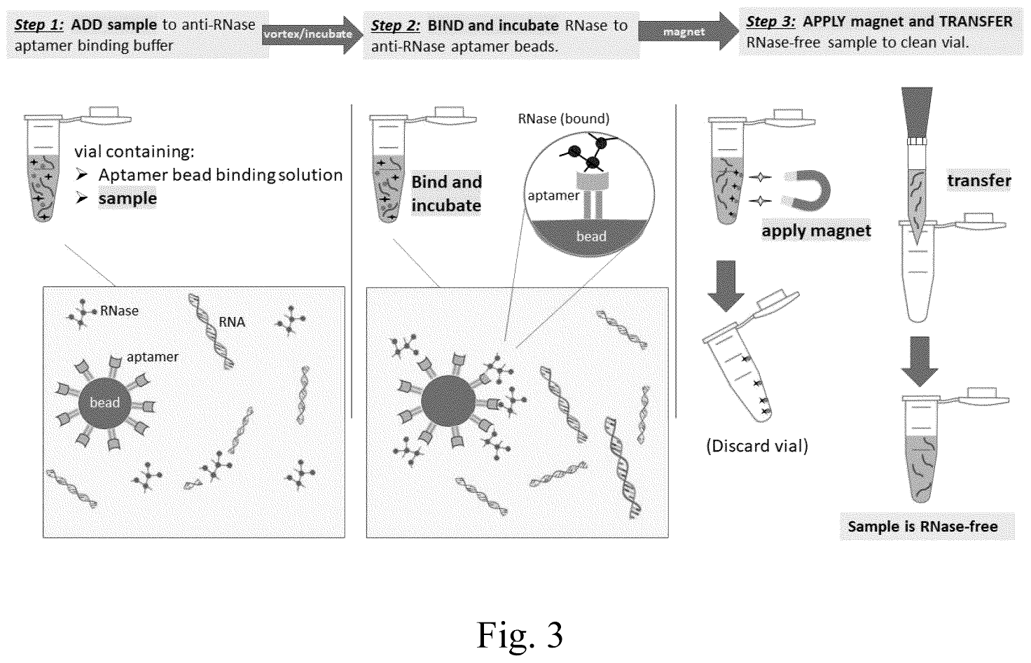

FIG. 3 is a schematical overview for an RNase Removal Kit. In this method, high affinity aptamers are covalently attached and immobilized by either 3′ prime or 5′ prime attachment to carboxy coated paramagnetic beads. The carboxy beads can be a fixed size or contain variable sizes ranging from 0.1 to 500 microns. The beads are included into an optimized binding buffer with pH ranging from 6.5-7.5. In this method, buffer containing immobilized aptamers affixed to paramagnetic beads are added to a biological sample. After addition, the mixture is vortexed and incubated for 5-10 minutes. During this incubation interval, RNase and DNase from collected biological samples will bind with high affinity to the aptamer present on the surface of the beads. The vial is then applied to a magnet or magnetic stand to allow beads to bind. The unbound aqueous mixture is transferred by pipettor into a new vial or microcentrifuge tube while remaining on the magnet stand. The old tube containing beads with bound RNase is discarded. The transferred eluate contains RNA and DNA from the biological specimen that is now free of nucleases and nuclease digestion. This quick RNase extraction kit can be used prefatory to expensive genomic applications such as RNA sequencing, RNASeq, single cell sequencing, and library preparation methods.

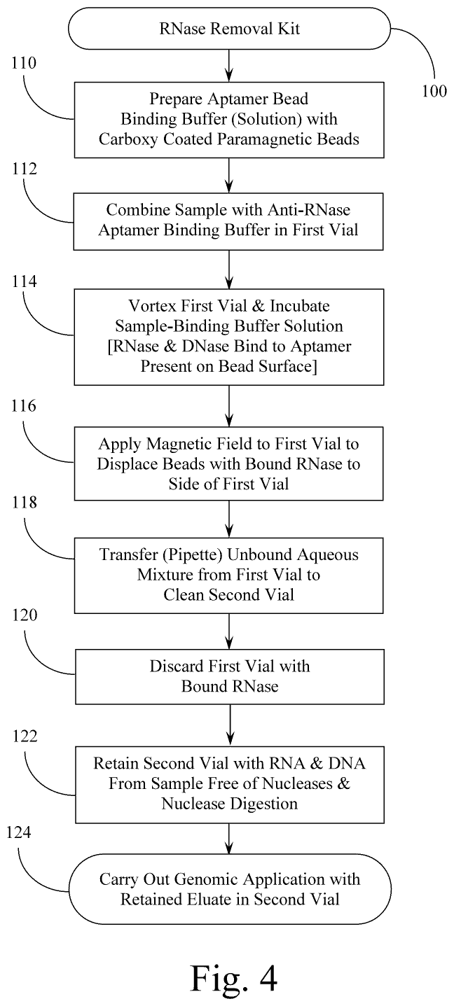

FIG. 4 is a method flowchart of the example method shown in FIG. 3 for using an RNase Removal Kit where high affinity aptamers are covalently attached and immobilized by attachment to carboxy coated paramagnetic beads. The process example of using RNase Removal Kit 100 begins at Step 110 with the preparation of aptamer bead binding buffer solution that includes carboxy coated paramagnetic beads. Step 112 comprises combining a collected biological sample with the anti-RNase aptamer binding buffer in a first vial. At Step 114, the first vial is vortexed and incubated to allow RNase and DNase to bind to aptamer present on the bead surfaces. Step 116 involves the application of a magnetic field to the first vial to displace the beads with bound RNase to one side of the first vial. The displacement at Step 116 allows for the collection and transfer of the remaining unbound aqueous mixture at Step 118 from the first vial to a second vial. Step 120 comprises discarding the first vial with the bound RNase. Step 122 comprises retaining the second vial with RNA and DNA from the biological sample free of nucleases and nuclease digestion. Finally, various genomic applications may be carried out at Step 124 with the preserved and stabilized eluate in the second vial.

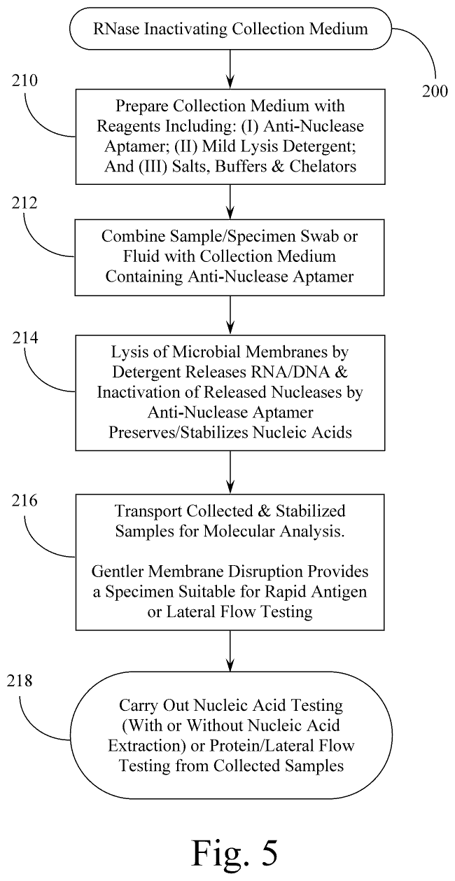

Reference is next made to FIG. 5 which is a method flowchart of a general approach for using an RNase Inactivating Collection Medium to stabilize a collected sample for transport and later use in nucleic acid and rapid antigen testing. The use of an RNase Inactivating Collection Medium 200 begins at Step 210 with the preparation of the collection medium with reagents that include one or more anti-nuclease aptamers, salts, and a mild lysis detergent. Step 212 involves combining the sample specimen swab or fluid with the collection medium containing the anti-nuclease aptamer(s). Step 214 involves the lysis of microbial membranes by the detergent which releases the RNA/DNA. This is followed by the inactivation of the released nucleases by the anti-nuclease aptamer(s) thereby preserving and stabilizing the nucleic acids. The collected and stabilized samples may then be transported (shipped) at Step 216 for later molecular or rapid antigen analysis. This process provides gentler membrane disruption and results in a specimen suitable for rapid antigen or protein/lateral flow testing or extraction-free (extraction-less) molecular testing (Step 218).

Table 1 (below) is a comparison of media types used for detection of Influenza A virus from a human clinical specimen. Clinical nasal swab was collected from a patient in Xtract-Free™ (LuJo BioScience Laboratory, San Antonio, Texas, USA) and Copan eNat® Molecular Transport Medium (MTM; Copan Diagnostics, Brescia, Italy). A 49-year-old male presented with fever (101.2° F.), aches/chills, and rhinorrhea with symptoms onset two days prior. In MTM media, collected specimens must be extracted and cannot be used for direct protein detection or in lateral flow tests. Conversely, specimens in Xtract-Free™ can be used for rapid antigen testing and molecular nucleic-acid tests including extraction-free (extraction-less) qRT-PCR. *Influenza detection performed using SARS-COV-2 & Flu A/B RNA STAR Complete (LumiraDx, London, UK), qPCR and Quick Vue Influenza A+B test (Quidel, San Diego, CA, USA).

TABLE 1

| | Xtract-Free ™ | |
|---|---|---|
| | Medium | eNat ® MTM |
| qPCR (with extraction) | Detected | Detected |
| qPCR (extraction-free) | Detected | Not Detected |
| RNA Star nucleic acid amplification* | Detected | Not Detected |
| Lateral flow rapid antigen test | Detected | Not Detected |

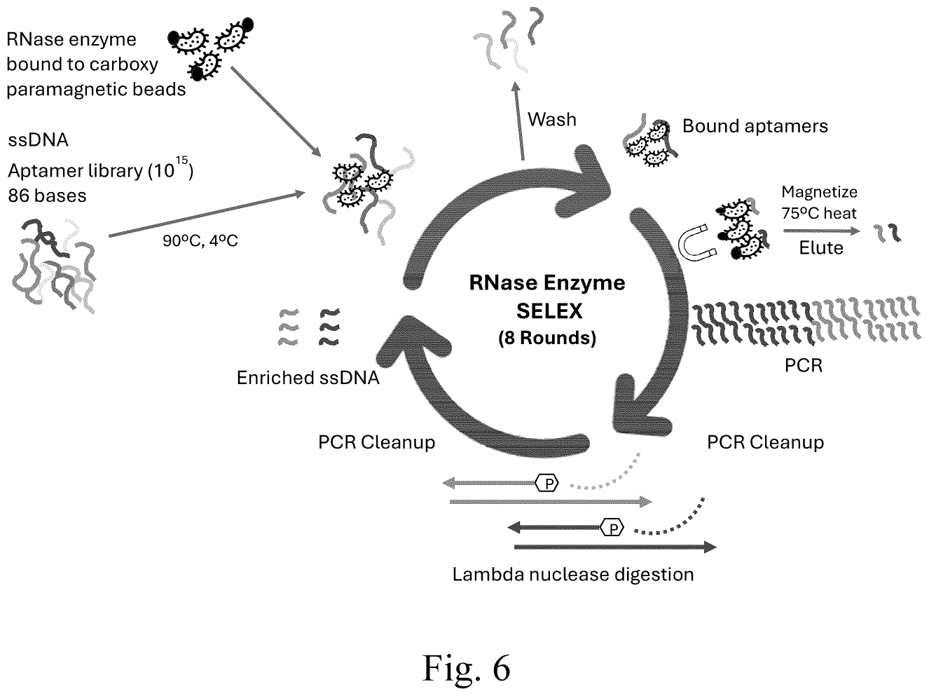

The anti-RNase aptamer library selection process is shown in FIG. 6. Aptamers are short nucleic acid oligonucleotides (typically 20-100 bases) that are functionally similar to antibodies and can recognize and bind to various biomolecular targets, including proteins. They are generated through a cyclic enrichment process known as Systematic Evolution of Ligands by Exponential Enrichment (SELEX). During SELEX, a massive library of random nucleic acid sequences (approximately 10¹⁵) is created through chemical synthesis and then exposed to an immobilized target molecule. Sequences that do not bind to the target are washed away, while those that do bind are amplified using polymerase chain reaction (PCR). The binding sequences are enriched through repeated cycles of incubation with the target, washing, and amplification. Eventually, a subset of sequences with high target binding affinity is selected.

### Experimental Methodology—In Vitro SELEX Selection of Anti-RNase A Aptamers

Referring again to FIG. 6, the synthetic aptamer library consisted of the following 86 nucleotides:

5′-TAG-GGA-ACA-GAA-GGA-CAT-ATG-AT-(N40)-TTG-ACT-
AGT-ACA-TGA-CCA-CTT-GA-3′

It was comprised of a 40-nt randomized region (10¹⁵ unique sequences) flanked on both sides by a 23-nt forward and reverse primer region for polymerase chain reaction (PCR). The aptamer library was synthesized by Integrated DNA Technologies (Coralville, USA). Additionally, SELEX-Forward, 5′-TAG-GGA-AGA-GAA-GGA-CAT-ATG-AT, SELEX-Reverse, 5′-TCA-AGT-GGT-CAT-GTA-CTA-GTC-AA-3′, and a 5′-phosphorylated reverse, 5′-Phos-TCA-AGT-GGT-CAT-GTA-CTA-GTC-AA-3′ were utilized (IDT, Coralville, USA). The aptamer library and all primers were diluted to 100 μM stocks. All primers were utilized at a 20 μM working dilution.

A total of 8 SELEX rounds were performed (see FIG. 6). A high SELEX stringency was employed for aptamers binding RNase by: 1) performing directly in Xtract-Free medium; and 2) at elevated ambient temperature (28° C.). For SELEX round 1, 100 μM aptamer library was diluted with nuclease-free water to a concentration of 2 nMol (10¹⁵ molecules) by adding 20 μL of ssDNA library to 80 μL Xtract-Free medium in a 0.2 PCR reaction tube. Aptamers were folded by incubation at 90° C. for 10 minutes and 4° C. for 15 minutes using a standard PCR thermocycler (ABI 2720, Foster City, CA). In a separate 1.5-mL sterile microcentrifuge, 100 μL of prepared carboxy beads with bound (immobilized) RNase A were added. Using the magnet stand, the beads were washed three times with 400 μL of SELEX binding buffer and resuspended in 100 μL of Xtract-Free collection medium. Combine the 100 μL of aptamer library in the 1.5 mL tube containing 100 μL of beads/Xtract-Free medium, pipette up and down several times to mix the solution, and incubate at elevated ambient temperature (28° C.) for 30 minutes with intermittent pipetting every 5 minutes. The mixture was placed on the magnet stand and washed twice with 400 μL of Xtract-Free buffer. The microcentrifuge tube was placed on a magnet stand and rotated 3-5 times manually during the washing steps to ensure bound aptamers were not dislodged from the beads. After two washes, the tube (with no fluid volume) was briefly removed from the stand to allow beads to migrate to the bottom of the 1.5 mL tube. The tube was placed back on the stand, and using a 10 μL pipettor, any trace fluid volume was removed from the tube. The tube was left open on the stand and allowed to airdry for 5 minutes. To elute bound aptamers, 200 μL of nuclease-free water (ThermoFisher, Waltham, MA) was added to the dried bead tube, pipetted up and down, and incubated at 70° C. for 7 minutes. After incubation, the microcentrifuge was briefly vortexed, placed on the magnet stand, and the fluid volume transferred to a new microcentrifuge tube.

### Polymerase Chain Reaction Amplification

Polymerase chain reaction (PCR) to amplify bound aptamers was performed during each SELEX round using a 50 μL total reaction volume of “MasterMix” consisting of: 32 μL nuclease-free water (ThermoFisher, Waltham, MA), 10 μL 5×PCR Buffer (Bioline, London, England), 2 μL forward and phosphate-labeled reverse primers (20 μM each), and 1 μL MyTaq polymerase (BioLine, London, England). The PCR reaction was performed in 0.2 mL PCR reaction tubes and consisted of an initial denature at 95° C. for 2 minutes, followed by 8 cycles of 94° C. for 30 seconds, 55° C. for 20 seconds, and 72° C. for 30 seconds. A final extension step was performed at 72° C. for 5 minutes. Positive and negative (no template) control reactions were included in each PCR run. A total of 10 μL of PCR product consisting of approximately 86-bp was visualized by UV illumination on a 2% agarose gel with ethidium bromide staining. Prior to visualization, gels were electrophoresed in TRIS borate EDTA (TBE) buffer at 90 V for 1 hour. The remaining 40 μL of PCR products were separated from primers and cleaned using the NEB Monarch PCR and DNA Cleanup Kit (NEB, Ipswich, MA) as specified in the user's manual.

### Lambda Nuclease Digestion

To recover ssDNA for subsequent SELEX selection rounds, purified PCR amplicons were subjected to lambda nuclease digestion (NEB, Ipswich, MA). Briefly, 40 μL of purified PCR product were added to 5 μL of 10× Lambda exonuclease buffer, 1 μL of enzyme, and 4 μL of nuclease-free water, gently pipetted to mix, and incubated at 37° C. for 30 minutes. After dsDNA-to-ssDNA digestion, the reaction was heat-inactivated at 75° C. for 10 minutes. ssDNA was purified using the NEB Monarch PCR and DNA Cleanup Kit and eluted in a final volume of 20 μL. The resulting SELEX product was used in subsequent rounds or stored at −20° C. until use.

### Next-Generation Sequencing of SELEX Aptamer Rounds

The ssDNA pools from SELEX rounds 1 to 8 (R1-R8) that bound to RNase were prepared for next-generation sequencing (NGS) analysis following an amplicon sequencing library preparation. In brief, PCR was used to attach 5′ Illumina adapter overhands to the SELEX Forward (Ill-Adapter-Selex-Forward (56 bp): 5′-TCG TCG GCA GCG TCA GAT GTG TAT AAG AGA CAG-TAG GGA AGA GAA GGA CAT ATG AT) and Reverse primer (Ill-Adapter-Selex-Reverse (57 bp): 5′-GTC TCG TGG GCT CGG AGA TGT GTA TAA GAG ACA G-TCA AGT GGT CAT GTA CTA GTC AA). PCR reactions were carried out in a total volume of 50 μL containing 32 μL of nuclease-free water (ThermoFisher, Waltham, MA), 10 μL of 5×PCR buffer (Bioline, London, England), and 2 μL of forward and reverse primers (20 μM each). A total of 5 μL of aptamer template ssDNA was included. Cycling conditions were 3 minutes at 95° C., followed by 11 cycles of 15 seconds at 95° C., 15 seconds at 55° C., 15 seconds at 72° C., and a final extension step for 2 minutes at 72° C. Due to the initial concentration of ssDNA in Round 1, only 8 cycles were used. The PCR products were checked using illumination on a 2% agarose gel stained with ethidium bromide. A second NGS-PCR used primers containing adapter sequences with Illumina-specific barcodes (i7 and i5) to attach the oligonucleotides to the flow cell. For the second PCR, reactions were carried out in a total of 25 μL reaction volumes containing 1× High-Fidelity Master Mix, 1 μM of NGS_PCR #2 primers, and 2.5 μL of the purified first NGS-PCR product. The cycling conditions were 30 seconds at 95° C., followed by 6 cycles of 10 seconds at 95° C., 30 seconds at 55° C., 30 seconds at 72° C., and a final extension step for 2 minutes at 72° C. The second NGS-PCR was performed using only 6 PCR cycles to attach barcodes. The PCR products were purified as described by the manufacturer's instructions using the Agencourt AMPure XP Purification System. The final DNA concentration for each product was determined using a Qubit fluorometer with the DNA Quantification Kit according to the manufacturer's instructions. The eight samples corresponding to R1-R8 were mixed in an equimolar ratio to a final concentration of 4 nM, and the final NGS library was clustered at 8 pM with 10% of the 8 pM PhiX internal control added to the run. Sequencing was done was performed on an Illumina MiSeq platform (San Diego, CA) using the MiSeq Reagent Micro Kit v2 (150 cycles) in paired-end mode. Sequencing data were demultiplexed using bcl-2fastq2 v2.20.

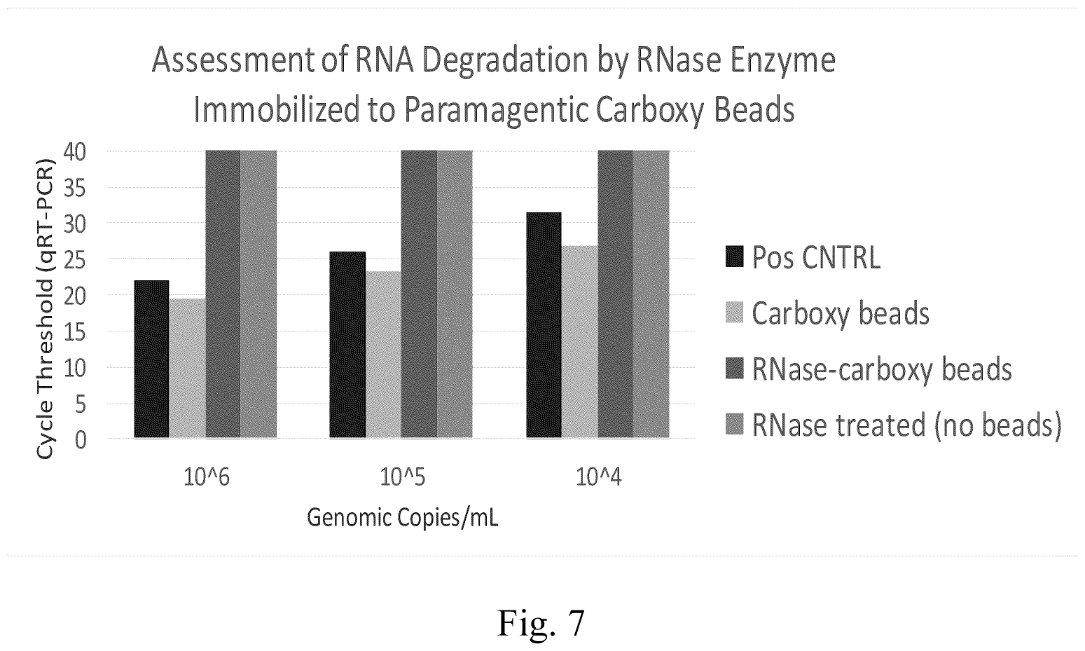

FIG. 7 demonstrates the successful immobilization (covalent binding) of RNase A protein to carboxy paramagnetic beads, using assessment of enzymatic activity by qRT-PCR detection. In this example, RNase A enzyme immobilization on surface-activated carboxy paramagnetic beads involved incubation with RNA, washing, and removal using a magnetic stand. Influenza A RNA incubated with RNase-bound beads was readily degraded (C_{T}=40), similar to equivalent RNase control reactions (Positive Control; RNase treatment), in comparison to a control containing beads without bound RNase (beads only). Three concentrations of SARS-COV-2 viral RNA as indicated. This experiment illustrates that using paramagnetic beads with bound (immobilized) RNase enzyme in conjunction with qRT-PCR can serve as an effective test system for evaluating RNase-inactivating aptamers (FIG. 7).

### Immobilization of RNase A Enzyme on Surface Activated Carboxy Paramagnetic Beads (FIG. 7)

Sera-Mag Carboxylate-Modified Magnetic Particles (Cytiva, Little Chalfont, United Kingdom) were used for immobilization of purified RNase A (Roche Diagnostics, Mannheim, Germany) by covalent binding according to the following procedure: A solution of RNase enzyme (3 mg/mL) was prepared by dilution in MES buffer (0.1 M, pH 5.0). The carboxy beads were allowed to acclimate to ambient temperature and mixed thoroughly prior to use. A total of 1 mL of beads (50 mg/mL) was added to 7.5 mL of nuclease-free water and mixed by pipetting. The solution was magnetized using a bead stand for 1 minute until clear, and the fluid volume was aspirated and discarded. A nuclease-free water wash was performed an additional two times to remove trace amounts of carboxy bead solution. For two times, a total of 7.5 mL of 0.1 M NaOH was added to the three-washed beads, mixed, placed on the magnetic stand for 1 minute, aspirated, and discarded. The beads were washed an additional two times with 7.5 mL of nuclease-free water to remove trace NaOH. A total of 7.5 mL of MES (0.1 M) was added to the beads, removed from the stand, and thoroughly mixed. In a sterile 15-mL Falcon tube, 200 mg of 1-ethyl-3-(3-dimethylaminopropyl) carbodiimide (EDC; Sigma, St. Louis, MO) and 100 mg of N-hydroxysuccinimide (NHS) were combined with 2 mL of MES (0.1 M; pH 5; Sigma, St. Louis, MO) and mixed thoroughly. Carboxy beads suspended in 7.5 mL of MES were added to the magnet stand, and the MES liquid was aspirated and discarded. To activate carboxy beads, 2 mL of EDC/NHS solution was added to the beads, vortexed briefly, and placed on a mini-tube rotator for 1 hour. The mixture was placed on the magnet stand for 1 minute, and the fluid volume was aspirated (pH 5), followed by a resuspension of the beads in 1 mL of MES (0.1 M; pH 5). To bind RNase to the beads, 1 mL of RNase protein (3 mg/mL) was added, mixed briefly, and placed onto the mini tube rotator for 1 hour. The mixture was placed on the magnet stand, and the fluid volume was removed and discarded by pipetting. The beads were resuspended in 2 mL of Trizma-HCl (0.1 M; Sigma, St. Louis, MO), mixed thoroughly, and placed on the mini tube rotator. After 1 hour, the solution was placed on the magnet stand for 1 minute, and the fluid volume was removed and discarded. Using the magnet stand, the beads were washed twice using 7.5 mL of PBS/0.1% Tween-20 (Sigma; St. Louis, MO). Finally, beads containing immobilized RNase enzyme (45 μg RNase to 45 mg beads) were resuspended in 7.5 mL PBS/0.1% Tween-20, mixed well, and stored at 4° C. until use.

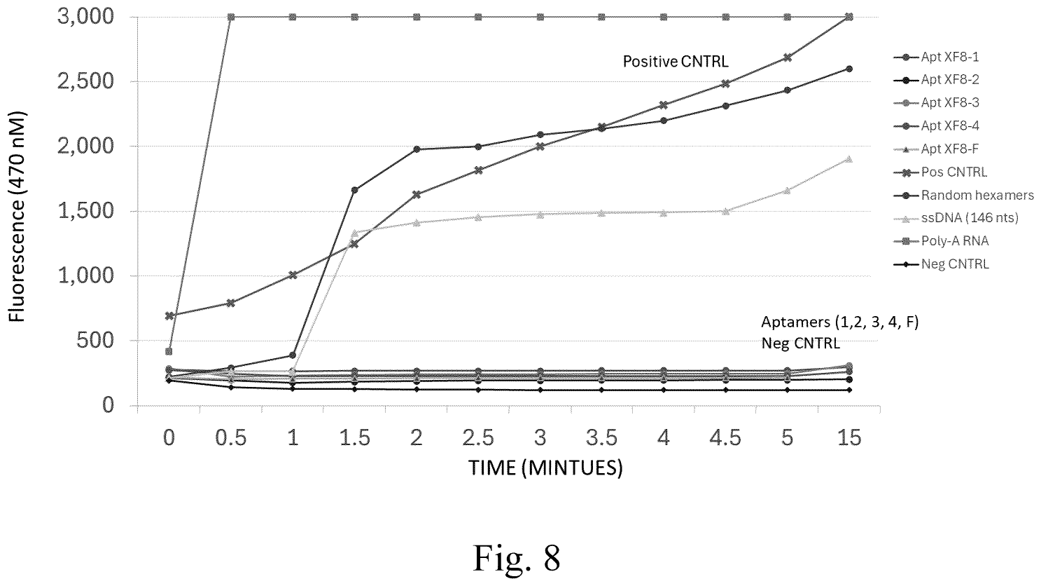

FIG. 8 depicts a 15-minute time course showing real-time fluorescence of enzymatic RNA degradation from RNase A enzyme. After only 15 minutes, ssRNA targets in the presence of the 5 anti-RNase aptamers exhibited little to no degradation and were similar to controls where no RNase was added. Conversely, reactions with random hexamers, a ssDNA (non-aptamer) sequence, or poly-A RNA incubated with RNase A showed increasing degradation as assessed by Qubit fluorometer readings. Positive and negative control reactions were included.

### Evaluating Anti-RNase Aptamers Using Substrate Nuclease Detection (FIG. 8)

Direct evaluation of anti-RNase digestion was conducted using the RNaseAlert Substrate Nuclease Detection System, following the manufacturer's guidelines (Integrated DNA Technology, Coralville, IA). The assay includes ssRNA with a 5′ fluorescein (reporter) and 3′ dark quencher. Intact RNA does not fluoresce, but RNase cleavage by RNase causes fluorescence, which is visible under UV light and by quantitative measurement with a fluorometer. The assay was carried out by adding 5 μL of 10× Buffer, 45 μL of 100 μM aptamer, and a final concentration of 20 μg/mL of RNase enzyme. After the addition of RNase, samples were immediately vortexed, and measurements at 470 nM were taken every 30 seconds for 15 minutes using a Qubit 4 Fluorometer (ThermoFisher, Waltham, MA). Additionally, visual inspection under UV light was conducted after 1 hour.

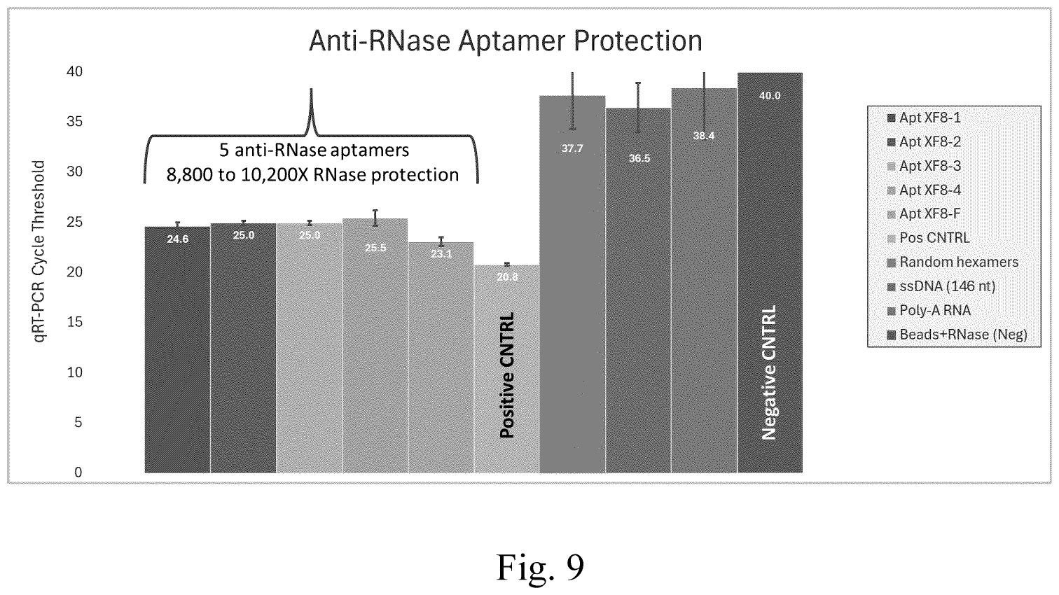

FIG. 9 is a qRT-PCR assessment of anti-RNase aptamer binding and inactivation of RNase enzyme when incubated with influenza A viral RNA (10⁶ copies/μL) for 15 minutes at 37° C. Compared to an equivalent concentration of viral RNA with no nuclease digestion, five aptamers (SEQ ID NO: 1-SEQ ID NO: 5) exhibited similar recovery values. When compared to the negative control (C_{T}=40) where no detection was noted, the aptamers showed 8,800-fold to 10,200-fold differences in detection. Other oligos, including random hexamers, a 146 nt ssDNA, and poly-A RNA, showed no RNase inactivation and were similar to negative controls. This experiment shows triplicate reactions with standard deviation displayed.

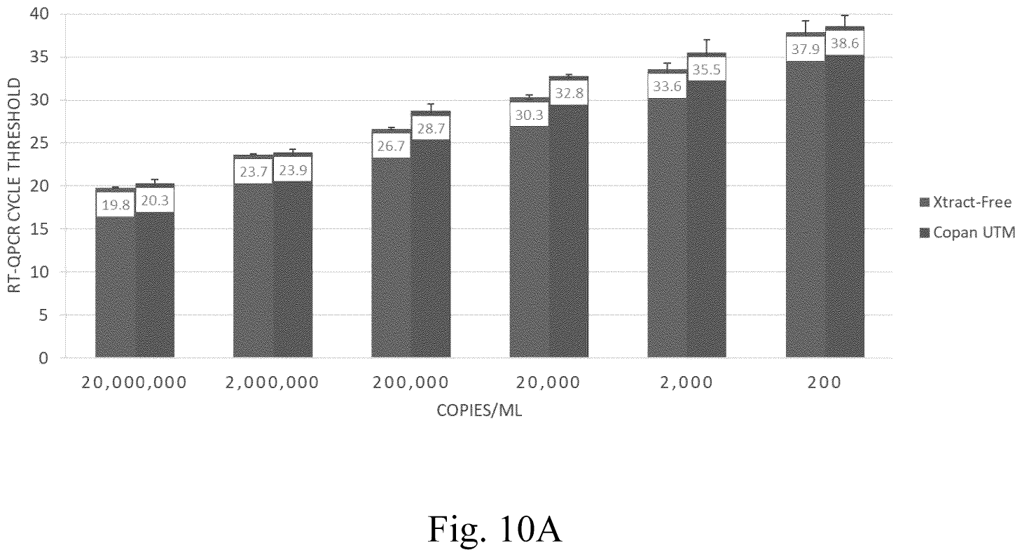

FIGS. 10A & 10B show a limit of detection (LOD) from quantified viral SARS-COV-2 RNA (vRNA) in a novel collection medium (Xtract-Free) or UMT is shown in FIGS. 10A & 10B. According to RT-qPCR, 5 of 5 replicates containing SARS-COV-2 RNA were detected from XF and UTM dilutions containing 2×10⁷ copies/mL to 2,000 copies/mL (5-logs). At the lowest dilution, i.e., 200 copies/mL (0.2 copies/μL), 4 of 5 replicates were detected in each medium. At 2,000 copies/mL, the lowest LOD dilution where 5 of 5 replicates were detected in Xtract-Free and UTM (average CDC qPCR C_{T} value: 33.6 and 35.5, respectively), 10 additional replicates were detected (data not shown). Average C_{T} values for vRNA in a 10-fold reduction series (6-logs) for each medium are shown (FIG. 10A).

As shown in FIG. 10B, positive results by LumiraDx's RSC assay are shown in comparison to respective C_{T} values by RT-qPCR. The RSC assay detected 1 of 5 (20%) replicate reactions at the lowest 200 copies/mL dilution. A total of 5 of 5 (100%) replicates were positive at 2,000 copies/mL using RSC.

A detailed methodology for FIGS. 10A & 10B is as follows. For testing comparison, the CDC 2019-Novel Coronavirus (2019-nCOV) Real-Time RT-PCR Diagnostic Panel was used as described (14). Briefly, using QIAamp RNA Viral Mini Kit (Qiagen, Hilden, Germany), 140 μL specimen was added to 560 μL Lysis Buffer AVL and subjected to spin-column viral RNA extraction with 60 μL final elution in Buffer AVE according to manufacturer's recommendations. For RT-qPCR detection of SARS-COV-2 RNA, the TaqPath 1-step RT-qPCR MM (ThermoFischer Scientific, Waltham, MA) was used with CDC's primers and probes targeting N1 and RNaseP on a QuantStudio 5 instrument (ThermoFisher, Waltham, MA). Positive and negative control reactions were included for each RT-qPCR run. During RT-qPCR analysis, clinical samples testing positive were recorded according to cycle threshold (C_{T}) value of N1 viral target. The RNaseP, a human gene target was included as an internal positive control for each specimen tested. Lower C_{T} values indicate a higher initial viral RNA concentration, with a value>40 indicating no amplification present. After initial qPCR testing, clinical specimens were stored at −80° C. until use.

A limit of detection (LOD) was performed using RT-qPCR and RNA Star Complete by 10-fold serial dilution of purified SARS-COV-2 viral RNA into each medium. For this experiment, 5 replicate reactions from each dilution were evaluated. At the lowest dilution where 5 of 5 replicates were detected, an additional 10 replicates were performed (data not shown).

### LumiraDx Testing

The SARS-COV-2 RNA Star Complete test (LumiraDx, London, UK) was performed as described. Briefly, for pre-processing, 24 μL of specimen was added to a 96-well optical plate containing 4.8 μL of Extraction Buffer, pipetted 10 times and briefly spun to bring contents down. To each reaction, 31.2 μL Reaction Mix (containing 10 μL of Salt, 1.2 μL of IC/P Mix, and 20 μL of Master Mix) was added to bring the total reaction volume to 60 μL/rxn. The plate was pipetted up/down 10 times and briefly spun before initiating run. Analysis was performed according to defined thermocycling parameters described (16) using a QuantStudio 5 instrument (ThermoFisher. Waltham, MA). The instrument run time for the RNA Star Complete assay is approximately 20 minutes. Statistical analysis to comparator test was performed using MEDCALC23 for determination of positive percent agreement (PPA), negative percent agreement (NPA), confidence intervals (CI), accuracy, and disease prevalence.

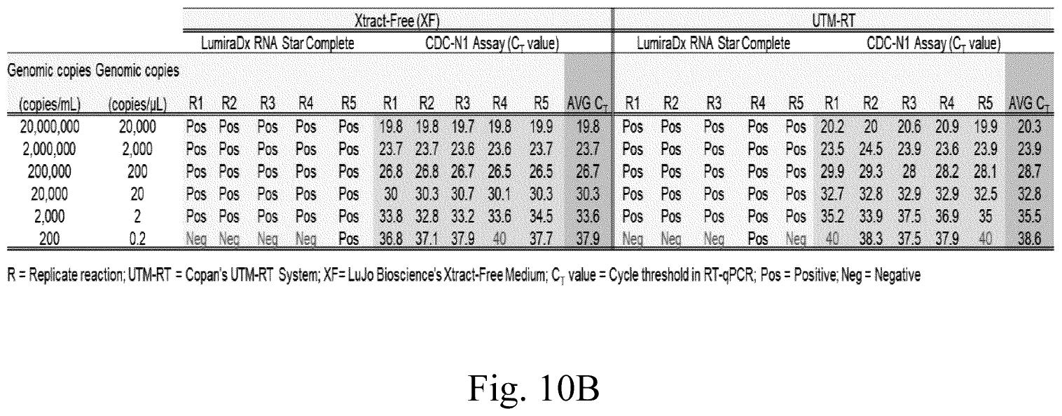

FIG. 11 is a human clinical study performed using Xtract-Free for sample collection and LurimaDx's RNA Star Complete molecular assay for car side SARS-COV-2 detection during the ongoing COVID-19 pandemic. Of 108 clinical specimens collected in Xtract-Free (46 true positive (TP) and 62 true negatives (TN) by RT-qPCR), 43 were positive and 65 were negative according to RSC. Positive percent agreement (PPA), defined as percentage of specimens testing positive among 46 true positive samples, was 93.9% (C.I.=83.1-98.7%) compared to ‘gold standard’ RT-qPCR. Negative percent agreement (NPA), defined as percentage of specimens testing negative among 62 true negative samples, was 100% (C.I.=94.2-100%). There were 3 false-negative (undetected) samples by RSC from previous positive samples where initial RT-qPCR C_{T} values were low, i.e., C_{T}>34.8, with one sample having a Cr value of 39.2. FIG. 11 summarizes clinical detection results of RSC according to PPA, NPA, and accuracy according to RT-qPCR analysis.

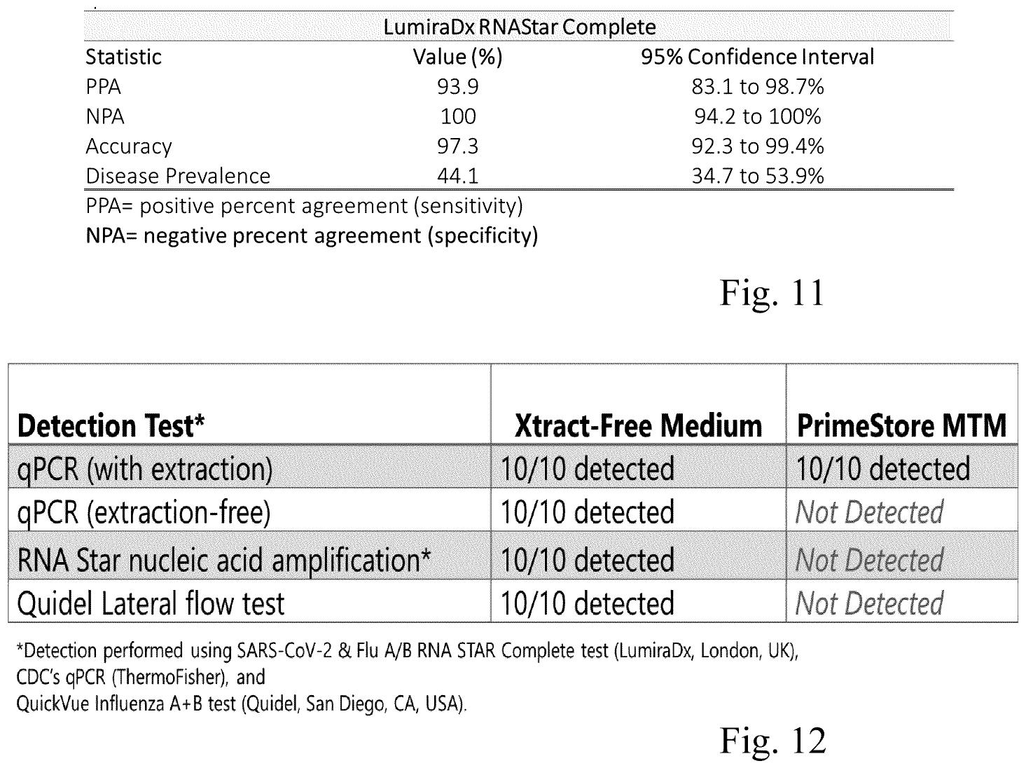

FIG. 12 demonstrates the capability to conduct multiple tests using samples from the same collection vial containing Xtract-Free medium. In this instance, 10 human clinical nasal samples were gathered in a vial with 1.5 mL of Xtract-Free. A second nasal sample was obtained from the same 10 human subjects and stored in PrimeStore MTM, a molecular transport medium. Unlike PrimeStore MTM, which only identified samples through qPCR post nucleic acid extraction, Xtract-Free medium successfully detected the SARS-COV-2 virus in all 10 samples using various methods: 1) directly in qPCR (without extraction), 2) with extraction and qPCR, 3) used directly (without extraction) in an isothermal molecular test, and 4) employed in a lateral flow test. The specimens were collected during the 2022-2023 season of the ongoing COVID-19 pandemic.

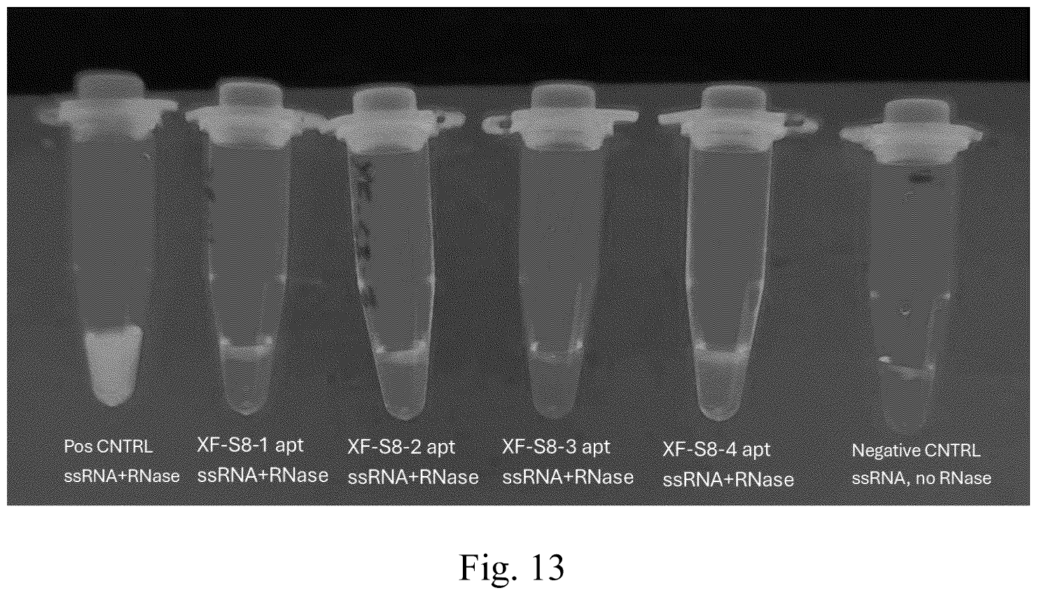

FIG. 13 is visual inspection of anti-RNase aptamers (XF-S8-1 to XF-S8-4) under UV transillumination at 5 days post incubation with RNase enzyme. The vials containing aptamer remain clear indicating that aptamer has bound to the active site of RNase A and inactivated its ability to enzymatically degrade RNA. Positive and negative control vials are shown.

An embodiment of this invention will employ the use of generated DNA or RNA aptamers as a single aptamer or combinations of DNA and RNA aptamers for targeting:
I. a DNA nuclease,
II. an RNA nuclease,
III. families and/or superfamilies of DNA nucleases,
IV. families and/or superfamilies of RNA nucleases,
V. combinations of DNA and RNA nucleases, families, and/or superfamilies.

For nuclease inactivation and subsequent RNA/DNA preservation/stabilization and recovery. The anti-nuclease aptamer will be supplemented at concentrations sufficient to be used as free-floating components in the aqueous mixture; or alternatively, immobilized onto beads in collection media for inactivating nucleases for the purpose of enhanced RNA/DNA stability and preservation of specimens at ambient or elevated temperatures. An embodiment and method of use for this invention where anti-nuclease aptamer(s) are utilized in sample collection:
I. directly as a component of a novel aqueous specimen collection medium,
II. included into existing specimen collection mediums (e.g., UTM/VTM, saline/PBS, MTM medium)

In another aspect of this invention, anti-nuclease aptamers are included into an optimized and novel ‘nuclease removal kit’ wherein anti-nuclease aptamer(s) are attached and immobilized to para-magnetic beads or with affinity resin spin columns to quickly and selectively remove contaminating nucleases for downstream purification and enhanced RNA/DNA recovery for downstream genomic applications including metagenomic, single cells analysis, NGS, and transcriptome analysis, particularly where miniscule amounts of RNA or DNA from samples are critical for nucleic acid detection and characterization.

In certain aspects of this invention, the aptamer or aptamers are short single-stranded oligonucleotides that range between 20 and 200 nucleotide bases capable of binding nucleases with high affinity and specificity wherein the target of the aptamer(s) is a DNA or RNA nuclease enzyme or ribozyme.

In more exemplary embodiments, the target of the aptamer(s) is the active site or sites on the DNA or RNA nuclease enzyme or ribozyme wherein the aptamer or aptamers target and inactivate DNA or RNA nucleases comprised of protein, or RNA, or both. In many, but not all cases, the target RNase and DNase molecules utilize water, (deoxy) ribose, ribose, inorganic phosphate, or the sidechains of Ser, Tyr or His as a nucleophile.

In one specific embodiment, the DNA or RNA anti-nuclease aptamer(s) bind to individual nucleases that are members of families or superfamilies, that include but are not limited to topoisomerases sequence-specific recombinases, metal ion-independent ribozymes, Holliday junction resolvases RNases, and DNases and RNases containing various atomic structures and catalytic mechanisms for degrading RNA and DNA polymers.

In another embodiment, DNA or RNA anti-nuclease aptamer(s) target DNase or RNase that are exonucleases, which remove one nucleotide at a time from the end of a strand at the 5′ to 3′ or 3′ to 5′ polarity, or alternatively, the DNA or RNA anti-nuclease aptamer(s) target DNase or RNases that are endonucleases, which cleaves a polynucleotide DNA or RNA chain by separating nucleotides other than the two end ones.

In a certain embodiment, DNA or RNA anti-nuclease aptamer(s) target and bind DNase or RNase enzymes that cleaves single-stranded or double stranded RNA or DNA, and the DNA or RNA anti-nuclease aptamer(s) target RNases, protein enzymes or ribozymes displaying several mechanistic cleavage activities that degrade RNA. In this embodiment, DNA or RNA anti-nuclease aptamer(s) target RNases that include protein enzymes RNase A, RNase B, RNase C, RNase H, RNase T1, RNase H2, RNase HP, RNase L, RNase I, RNase II, RNase III, and RNase IV.

In a method for use, DNA or RNA aptamer(s) binds specifically to nucleases, thus protecting RNA and DNA obtained from a collected primary human, environmental, or veterinary sample. In a more specific method, the collected primary specimen is a respiratory specimen obtained from a nasal wash, sputum, oral secretion, nasopharyngeal (NP) swab, nasal swab, or oral swab collection. Alternatively, the method described herein is also applicable for other human clinical samples including but not limited to buccal, urine, vaginal, penal, bile, serum, blood, seminal, fecal, or liquid biopsy samples. Furthermore, collected sample types that would benefit from this invention and the method of use include: i) a veterinary sample of animal and avian origin including but not limited to cloacal, buccal, urine, vaginal, penal, bile, serum, blood, fecal, or liquid biopsy samples, and ii) environmental samples such as air, water, soil, biological materials, and wastes.

In one aspect of this invention, DNA or RNA nuclease-inactivation aptamer or aptamers are supplemented into normal saline, phosphate buffered saline, Hank's medium, Minimal Essential Medium, (MEM) or commercial and common VTM, UTM, MTM used for sample collection.

In another aspect, DNA or RNA nuclease-inactivation aptamer or aptamers are supplemented into DNA or RNA extraction or purification kits that employ spin columns or magnetized beads or used in the kit's respective bead-washing solutions, binding solutions, lysis buffers, washing buffers, or elution solutions.

In an additional use for this invention, DNA or RNA nuclease-inactivation aptamer or aptamers are supplemented into next-generation sequencing library preparation kits, subtraction kits, RNA-Seq kits, DNA-Seq kits, transcriptome kits, epigenetic kits, ribodepletion kits, CRISPR kits for genomic analysis of RNA or DNA using next-generation sequencing, sanger sequencing, fluorescent chain-termination sequencing, metagenomics, epigenomics, or nanopore sequencing methodologies.

Alternatively, DNA or RNA nuclease-inactivation aptamer or aptamers are supplemented into pathogen detection kits, pre-formulated ‘master-mix’ blends used for nucleic acid testing of viruses, fungal, bacterial or human targets using traditional PCR, RT-PCR, qPCR, RT-qPCR, digital drop PCR, isothermal amplification or non-isothermal amplification methods; or augmented into forensic, fungal, plant-based, veterinary, or environmental sampling and collection kits.

In one aspect of this invention, DNA or RNA nuclease-inactivation aptamer or aptamers are supplemented into solutions, carrier matrices or lyophilization formats employing RNA or DNA target controls and quantification standards used in nucleic acid testing applications.

In one method, DNA or RNA aptamers targeting nucleases contain unmodified nucleotides; alternatively, anti-RNase and DNase aptamers may contain one or more modified nucleotide bases, unnatural bases, mirror-image bases (spiegelmers), modified sugar rings, or modified phosphates. Examples of such aptamer modifications include: 2′-F, 2′-NH2, phosporothioate (PS), 2-Ome, LNA (bridging 2″ and/or 4′ ribose covalently), phosphoorodiothioate (PS2) linkages, heavy bases, modification at the 5-position of the heterocyclic base (e.g., benzyl, naphthyl, indole changes). Additionally, modifications may include specialized changes for aptamer stability including inter-nucleotide linkage with 3′-3′ and 5′-5′ capping in the terminus, inverted thymidine, 2′ substitutions and phosphodiester linkage replacement, 2′-fluoropyrimidines, 2′-O-methylpurines, 2′-ribopurine, 2′-O-methyl, L-ribonucleic acid, D/L-Iso-nucleoside modifications, or circular linkage; or specialized conjugations to include cholesterol, lipid species, diacylglycerol (DAG), PEGylation, carbohydrate, or protein peptide species.

In one method, anti-nuclease DNA or RNA aptamers are used to bind nucleases to stabilize, protect, and shield degradation of viral RNA and DNA obtained from influenza viruses, Sudden Acute Respiratory Syndrome (SARS), coronaviruses including COVID-19, adenoviruses, RSV, parainfluenza, rhinoviruses, and Ebola from specimens collected in medium for sample collection; or any one or combination of clinical bacterial-derived RNA and DNA from Streptococcus pneumoniae, Mycoplasma pneumoniae, Moraxella catarrhalis, Streptococcus pyogenes, Haemophilus influenzae, and Chlamydophila pneumoniae, Mycobacterium tuberculosis, mycobacterium Tuberculosis antibiotic resistance genes, and various mycobacterium spp.

In similar fashion, anti-nuclease DNA or RNA aptamers are used to stabilize, protect, and shield degradation of bacterial and viral RNA and DNA from veterinary specimen collection medium and detection kits used for bluetongue virus (BTV) African Swine Fever (ASF), Newcastle Disease, foot and mouth disease, rabies, avian influenza, West Nile fever, sheep/goat pox, lumpy skin disease, Rift Valley Fever (RVF), and Classic Swine Fever (CSF).

The present invention is also applicable for use in genomic analysis kits prefatory to sequencing and analysis wherein anti-nuclease DNA or RNA aptamers are used to stabilize, protect, and shield degradation for genomic transcriptome analysis, whole-genome, metagenomic, and next-generation sequencing; alternatively, anti-nuclease aptamers would be highly beneficial for inclusion in enzymatic reactions kits and buffers for transcription analysis kits, CRISPR kits, RNA-Seq, recombination analysis, and CRSIPR-based detection kits, T7-transcription kits, T3-transcription kits, mRNA synthesis kits, plasmid preparation kits, transcription purification kits, and protein translation chemistry kits.

In one embodiment, anti-nuclease DNA or RNA aptamers are used to stabilize, protect, and shield degradation and enhance preservation of RNA/DNA targets used in therapeutic vehicles, matrix medium, or solutions used for targeted delivery via liposomal, viral or artificially including RNAs, drugs, genomic delivery systems, or chemical preparations for use in diagnostic or therapeutic treatment, detection, recombination, genomic repair, in vitro, in vivo, ex-in vivo methods.

In another embodiment, anti-nuclease DNA or RNA aptamers are used to stabilize, protect, and preserve nucleic acid polymers in specimen collection media and culture media used for collecting, transporting, and processing specimens for nucleic acid amplification and sequencing from viruses, bacteria, plant, and fungal sources. In this approach, anti-nuclease DNA or RNA aptamers are used in collection media, nucleic acid extraction and purification kits, and in detection and genomic characterization kits used for animal and human genetic variation consisting of an allele, multiple alleles, or genetic abnormality associated with a genetic disease or disorder.

In a more specific embodiment, anti-nuclease DNA or RNA aptamers are used in collection medium for inactivating nucleases prior to specimen transport, pre-processing with or without RNA/DNA extraction, and detection and genetic characterization kits such as rapid, non-isothermal nucleic acid amplification method intended for the qualitative detection of nucleic acid from SARS-COV-2, Mycobacterium tuberculosis, and influenza viruses in upper respiratory swabs, oral fluids, oral and nasal washings or sputum collected from individuals.

Likewise, anti-nuclease DNA or RNA aptamers are used in a novel transport medium, or in UTM/VTM, PBS, saline solutions and MTM for the purpose of inactivating nucleases from primary specimens originating from respiratory, urine, or genital collection from humans, chickens, or other mammals for detection of pathogenic viruses and bacteria.

In this embodiment, a novel transport medium contains an RNASE enzyme inactivating aptamer or combination of aptamers. Furthermore, the novel transport medium contains an anti-RNASE aptamer and includes one or more gentle nonionic detergents such as Tween-20, Tween-80, sodium lauryl isethionate in concentrations sufficient for lysis of membranes and subsequent inactivation nuclease inactivation from viruses and bacterial and human cells. In addition to inclusion of aptamers, a novel collection medium is substantially different from other collection medium, particularly MTMs and VTM/UTM medium because the formulation contains:
i. no guanidine-based compounds,
ii. no N-Lauroylsarcosine sodium salt or sodium dodecyl sulfate,
iii. no Triton-X or Triton-like derivatives,
iv. no harsh chaotropic agents or reducing agents (DTT, DMF, DMSO, etc.),
v. no flammable alcohols (ethanol, methanol, etc.),
vi. no PBS, balanced (HANK's) salt mixtures, or high-saline solution.

Thus, a novel collection medium described here that is substantially different from MTMs is non-corrosive, non-flammable, eco-friendly, and does not produce toxic byproducts such as cyanide gases upon cleanup with bleach-based compounds. Furthermore, a novel transport medium contains an anti-RNASE aptamer or collection of aptamers that can be used for collected samples and specimens without extraction (extraction-less) or purification (i.e., extraction-less) directly with PCR and other NAT applications such as qPCR, isothermal amplification, and non-isothermal methods. Finally, a novel transport medium contains a mixtures or chemical reagents optimized for (direct) synergistic use with NAT detection ‘master mixes’, buffers, and single-cell, RNA-Seq, and other library preparation reagents and pre-processing methods for downstream sequencing, characterization and genotyping analysis. Finally, a novel transport medium containing an anti-RNASE aptamer will shield, protect, and stabilize RNA and DNA polymers at ambient temperature or elevated temperatures for extended periods.

In a more specific embodiment of the above method and formulation, the novel transport medium contains:
i. an anti-RNASE aptamer targeting the active site of RNASE A (RNASE I), and a combination of chemicals; and
ii. an optimized blend of sodium acetate, sucrose, betaine, glycerol, Tween-20, a buffering agent (e.g., TRIS), and 0.5 Molar EDTA for microbial lysis, inactivation of nucleases, and preservation of nucleic acids, proteins, and antigens for extended periods and at elevated temperatures.

An anti-RNase aptamer whose anti-RNase binding and inactivation properties were optimized to function in elevated temperature and high salinity matrices and reagent blends; specifically, aptamers enriched in the formulation for a sample collection medium described herein.

Furthermore, in one method of use, a novel transport medium containing an anti-RNASE aptamer or combination of aptamers is included with an optimized formulation composition that performs synergistically and without additional laborious or time-consuming steps (e.g., nucleic acid extraction, high heat incubation, proteinase-k addition, etc.) for direct addition into and for use with testing methodologies that include:
i. nucleic acid testing including PCR and non-isothermal amplification;
ii. rapid antigen or lateral flow testing of protein targets;
iii. ELISA-based protein tests;
iv. Genomic sequencing and analysis such as next-generation sequencing;
v. high-throughput photometric and ion-elective electrode (ISE) determinations; and
vi. point-of-care or laboratory based electrochemical detection methods.

In this aspect of the invention, a novel collection medium is used for collection of primary specimens for the purpose of: direct pathogen detection or subtyping, detecting and characterizing pathogens or mutations conferring drug resistance to an antibiotic, antifungal, or a chemotherapy; or for detection and characterization of mutations and genomic signatures for characterizing chronic disease detection including human cancer, Alzheimer's, heart disease, prenatal diseases, autoimmune disorders, and neurodegenerative disorders.

In another embodiment of this invention, anti-RNASE or DNASE aptamer is covalently or otherwise bound and immobilized by 5′ or 3′ attachment to magnetic beads or particles, or to binding resins and used in affinity columns or paramagnetic beads for separation of nucleases from collected specimens, biological preparations for purification, or genomics applications and kits intended for characterization of nucleic acids from collected samples or in genomic library preparations and RNA sequencing workflows.

In another embodiment, anti-RNase or DNase aptamers are supplemented into reagents and preparations containing quantified RNA or DNA controls, targets, standards, or total genomic biobanked/archived pathogens intended for downstream nucleic acid testing and genomic characterization.

In the embodiment of this invention, anti-RNASE or DNASE aptamer(s) reduce the degradation of RNA or DNA in a sample a minimum of 5-fold (5×) or greater compared to equivalent untreated controls. Importantly, anti-RNase or DNase aptamer(s) added to collection medium, used in removal kits, or supplemented to buffered solutions for genomic detection and analysis will:
i. Enhance nucleic acid binding and recovery to silica dioxide beads or binding columns,
ii. Prevent, inactivate, and neutralize degradation of RNA/DNA via enzymatic activity by nucleases in collected samples.

The present invention overcomes the problems and disadvantages associated with reagent compositions and methods used in VTM/UTM, MTM, and common saline for collecting, transporting, and storing biological samples preferably for later diagnostic or genomic characterization.

One embodiment of the invention is directed to a novel collection composition comprising one or more aptamers for nuclease inactivation, one or more salts, one or more sugars, one or more buffers, one or more detergents, and one or more proteins. The composition does not contain guanidine-based compounds, reducing agents, alcohols, or other chemically restrictive or environmentally harsh reagents, pollutants, or toxic substances found in MTM compositions.

Ideally, the salts in the composition include potassium chloride (KCl), calcium chloride (CaCl₂)), magnesium sulfate (MgSO₄), magnesium chloride (MgCl₂), lithium chloride, sodium chloride (NaCl), sodium phosphate dibasic (Na₂HPO₄), or a combination of these. The sugars in the composition may include disaccharides, oligosaccharides, sucrose, fructose, glucose, dextrose, trehalose, galactose, ribose, deoxyribose, maltose, lactose, or a combination thereof. The buffer used is preferably Tris-HCl.

In a functional composition, the collection medium contains betaine, a reagent known to enhance the amplification of GC-rich stretches of nucleic acid. The composition should ideally have a final pH between 7.0 and 7.4, with a preference for 7.2 after adjustment using HCl.

Another embodiment of the collection medium composition includes: one or more chloride salts, one or more sodium salts, one or more ionic or non-ionic detergents, one or more chelators, and one or more lithium salts. Ideally, chloride salts consist of potassium chloride (KCl), sodium chloride (NaCl), or a combination of both. The sodium salts preferably include sodium acetate, sodium phosphate, sodium phosphate dibasic (Na₂HPO₄), or a combination of these. The detergents should include a TWEEN, like TWEEN 20, TWEEN 80, or a combination of both. The chelators should consist of ethylene glycol tetraacetic acid, hydroxyethylethylenediaminetriacetic acid, diethylene triamine penta acetic acid, N, N-bis(carboxymethyl)glycine, ethylenediaminetetraacetic, EGTA, HEDTA, DTPA, NTA, EDTA, potassium citrate, magnesium citrate, ferric ammonium citrate, citrate anhydrous, sodium citrate, calcium citrate, or ammonium citrate, or a combination of these.

In a preferred embodiment, a novel collection medium, substantially different from VTM/UTM and MTM, will contain a synergistic profile of non-toxic and environmentally friendly reagents that include one or more free-floating or immobilized aptamers generated for optimal activity within the chemical profile and at elevated ambient temperatures (22-30° C.). This design ensures the aptamers are effective at inactivating nucleases from collected samples within the collection medium and at temperatures at or above ambient. The overall composition of the medium, in contrast to VTM/UTM and MTM, enables the same collected sample to be used in multiple downstream applications such as qPCR, sequencing, and point-of-care tests like rapid antigen or lateral flow assays. Importantly, the medium will contain reagents optimized for use with or without nucleic acid extraction (i.e., extraction free PCR).

An exemplary composition of a novel collection medium is optimized to perform: 1) with or without a need for nucleic acid extraction (extraction-free) for molecular detection, 2) directly with protein detection including lateral flow, ELISA, and rapid antigen assays, 3) inactivation of nucleases using anti-nuclease aptamers, 4) gentle disruption of cellular and microbial membranes, 5) preservation of nucleic acids for extended periods at ambient temperature, 6) functionality within a eco-friendly composition of reagents that operate outside of toxic, caustic, and corrosive chemicals in common MTM's such as guanidine-based chemicals (e.g., guanidine thiocyanate), reducing agents (e.g., N-Lauroylsarcosine sodium salt), alcohols and Triton-derived (e.g., Triton-X) reagents.

An exemplary composition of a novel collection medium containing anti-nuclease aptamer(s) contains a reagent profile added in the following order and consisting of the final concentrations:
i. deionized, distilled and nuclease-free water
ii. Tris(hydroxymethyl)aminomethane (TRIS) buffer at a preferential final concentration of 1 to 100 mM; but substantially exemplary at 10 mM
iii. one or more anti-nuclease DNA or RNA aptamers at a preferential final concentration of 1 to 500 μM; but substantially exemplary at 100 μM
iv. sodium acetate at a preferential final concentration of 1 to 100 mM; but substantially exemplary at 1.22 mM
v. sucrose at a preferential final concentration of 1 to 100 mM; but substantially exemplary at 2.92 mM
vi. betaine at a preferential final concentration of 1 to 100 mM; but substantially exemplary at 45 mM
vii. glycerol at a preferential final concentration of 0.5 to 5%; but substantially exemplary at 0.8%
viii. tween-20 at a preferential final concentration of 0.1 to 5%; but substantially exemplary at 0.25%
ix. Ethylenediaminetetraacetic acid (EDTA) at a preferential final concentration of 0.1 to 2 mM; but substantially exemplary at 0.2 mM
x. hydrochloride (HCl) added a 10 Normal sufficient to adjust the pH of the final solution to 7.0 to 7.4; but substantially exemplary at a final pH of 7.2.

Using the method for enriching aptamers as described in FIG. 6, the following sequences (SEQ ID NO: 1-SEQ ID NO: 5) are presented in Table 2 (Selected anti-RNase Aptamer Sequences). These sequences represent select anti-RNase aptamers (single-stranded DNA, 86 nucleotides) consisting of a 23-nucleotide forward primer sequence, a 40-nucleotide random region (N40 region), and a 23-nucleotide reverse primer sequence. These aptamers have been shown to bind to and inhibit the RNase A enzyme. Note: Underline=forward and reverse primer sequence; Bold=40 nucleotide variable aptamer sequence.

| ID | Name | Sequence (5′-3′) |
|---|---|---|
| SEQ ID | XF-S8-1 | TAGGGAAGAGAAGGACATATGATGCTGCCGACGATT |
| NO: 1 | | GACTAGTACATGACCACTTGACTGTCTTTGACTAGT |
| | | ACATGACCACTTGA |
| SEQ ID | XF-S8-2 | TAGGGAAGAGAAGGACATATGATAGGGTGAGCATCC |
| NO: 2 | | GCATAACAATAGTGCTGTTTAGTTGGCTTGACTAGT |
| | | ACATGACCACTTGA |
| SEQ ID | XF-S8-3 | TAGGGAAGAGAAGGACATATGATCTAACTTGAATAA |
| NO: 3 | | ATACCAGCACCAGACTGCCCGCGTTTCTTCACTAGT |
| | | ACATGACCACTTGA |
| SEQ ID | XF-S8-4 | TAGGGAAGAGAAGGACATATGATGGACGGGTATACA |
| NO: 4 | | CTAGACAACAACAAGGAACACTCTTTCTTGACTAGT |
| | | ACATGACCACTTGA |
| SEQ ID | XF-S8-F | TAGGGAAGAGAAGGACATATGATGCTTTCCATGTCG |
| NO: 5 | | TTATCCTAGGGGCTGTTAGCTAATTTCTTGACTAGT |
| | | ACATGACCACTTGA |

TABLE 2

Although the present invention has been described in connection with certain exemplary embodiments, those skilled in the art will recognize additional embodiments not described in detail that still fall within the spirit and scope of the invention.

### Definitions

In the present invention, the following terms have the following meanings:

The term “aptamer”, as used here, refers to oligonucleotides that mimic antibodies in their ability to act as ligands and bind to analytes. In one embodiment, aptamers consist of natural DNA nucleotides, natural RNA nucleotides, modified DNA nucleotides, modified RNA nucleotides, or a combination of these.

The terms “selected library” or “selected aptamer library”, as used here, refer to a collection of aptamer sequences that have been exposed to a target, such as a protein or enzyme (e.g., RNase A), through a process known as aptamer selection. A selected library exhibits the characteristic that at least subset of sequences observed in the initial SELEX rounds are observed again in subsequent selection round against the same target.

As used herein, “sample” and “specimen” are interchangeable terms and include anything containing or presumed to contain a molecule target of interest. This may be matter containing nucleic acid, protein, or other biomolecule of interest. The term “sample” can encompass a microbe, virus, cell, tissue, or population of one or more of the same that includes nucleic acids (genomic DNA, mRNA, cDNA, RNA, protein, other cellular molecules, etc.). The terms “nucleic acid source,” “sample,” and “specimen” are used interchangeably herein in a broad sense and are intended to encompass a variety of biological sources that contain nucleic acids, protein, one or more other biomolecules of interest, or any combination thereof. Exemplary biological samples include, but are not limited to, whole blood, plasma, serum, sputum, urine, stool, white cells, buffy coat, swabs (including, without limitation, buccal swabs, throat swabs, vaginal swabs, urethral swabs, cervical swabs, rectal swabs, lesion swabs, abscess swabs, nasopharyngeal swabs) urine, stool, sputum, tears, mucus, saliva, semen, vaginal fluids, lymphatic fluid, amniotic fluid, spinal or cerebrospinal fluid, peritoneal effusions, pleural effusions, exudates, punctuates, epithelial smears, biopsies, bone marrow samples, fluid from cysts or abscess contents, synovial fluid, vitreous or aqueous humor, eye washes or aspirates, pulmonary lavage or lung aspirates, and organ or tissue samples. A specimen or sample may also refer to an environmental sample such as surface collection, sewage waste, water, and air. A specimen or sample may also be of veterinary or agricultural origin such as a cloacal or hog-derived sample. Samples and specimens may be derived from eukaryotic, prokaryotic, fungal, plant, and viral sources. In some embodiments, the sample may be, or be from, an organism or microbe that acts as a vector, such as a mosquito, tick, insect, or bacteriophage vector.

### REFERENCES

Krafft A E at al., “Evaluation of PCR Testing of Ethanol-Fixed Nasal Swab Specimens as an Augmented Surveillance Strategy for Influenza Virus and Adenovirus Identification,” Journal of Clinical Microbiology, vol. 43 (4), (2005): 1768-1775.
Chomezynski, P. and Sacchi, N., “Single-Step Method of RNA Isolation by Acid Guanidinium Thiocyanate-Phenol-Chloroform Extraction,” Anal. Biochem., (1987) 162:156-9.
Daum L T, Worthy S A, Yim K C, Nogueras M, Schuman R F, Choi Y W, Fischer G W. A clinical specimen collection and transport medium for molecular diagnostic and genomic applications. Epidemiol Infect. (2011); 139(11):1764-73.
Adachi T, Nakamura Y. Aptamers: A Review of Their Chemical Properties and Modifications for Therapeutic Application. Molecules. (2019): 21; 24(23):4229.
Zhang Y, Lai B S, Juhas M. Recent Advances in Aptamer Discovery and Applications. Molecules. (2019): 7; 24(5):941.
Dunn, M., Jimenez, R. & Chaput, J. Analysis of aptamer discovery and technology. Nat Rev Chem 1, (2017): 0076.
Adachi, and Nakamura. Aptamers: A Review of Their Chemical Properties and Modifications for Therapeutic Application. Molecules. (2019): 24; 4229.
“AgPath-ID One-Step RT-PCR Kit,” Applied Biosystems, available at http://www.abion.com/techlib/prot/bp-1005.pdf (last visited Aug. 24, 2009).
“Collecting, Preserving, Shipping Specimens for the Diagnosis of Avian Influenza (H5N1) Virus Infection: Guide for Field Operations,” WHO/CDS/EPR/ARO/2006.1 (2006).
Blow, et al., “Viral Nucleic Acid Stabilization by RNA Extraction Reagent,” J. of Virol. Meth., 150, pp. 41-44 (Apr. 2, 2008).
Fischer G W and Daum L T. U.S. Pat. No. 9,388,220-B2-Immunogenic Compositions and Methods. Priority Date: Aug. 27, 2007.
LumiraDx SARS-COV-2 RNA STAR Complete. LumiraDx Instruction for Use. Available at: https://www.lumiradx.com/assets/pdfs/fast-lab-solutions/sars-cov-2-rna-star-ifu-for-ruo-ous.pdf?v=1
Daum L T, Rodriguez J D, Ward S R, Chambers J P. Extraction-Free Detection of SARS-CoV-2 Viral RNA Using LumiraDx's RNA Star Complete Assay from Clinical Nasal Swabs Stored in a Novel Collection and Transport Medium. Diagnostics. 2023 Sep. 21; 13(18):3010.
Daum L T, Choi Y W, Worthy S A, Rodriguez J D, Chambers J P, and Fischer G W. Molecular Transport Medium for Collection, Inactivation, Transport, and Detection of Mycobacterium tuberculosis. The International Journal of Tuberculosis and Lung Disease. 2014 July; 18(7):847-9.
Daum L T, Fourie P B, Bhattacharyya S, Ismail N A, Gradus S, Maningi N E, Omar S V, Fischer G W. Next-generation sequencing for identifying pyrazinamide resistance in Mycobacterium tuberculosis. Clinical Infectious Diseases. 2014 March; 58(6):903-4.
Daum L T, Fischer G W, Sromek J, Khubbar M, Hunter P, Gradus M S, Bhattacharyya S. Characterization of multi-drug resistant Mycobacterium tuberculosis from immigrants residing in the USA using Ion Torrent full-gene sequencing. Epidemiology and Infection. 2013 Sep. 27:1-6.
Sutter D E, Worthy S A, Hensley D M, Maranich A M, Dolan D M, Fischer G W, Daum L T. Performance of five FDA-approved rapid antigen tests in the detection of 2009 H1N1 influenza A virus. Journal of Medical Virology. 2012 November; 84(11):1699-702.
Daum L T, Rodriguez J D, Worthy S A, Ismail N A, Omar S V, Dreyer A W, Fourie P B, Hoosen A A, Chambers J P, Fischer G W. Next-generation ion torrent sequencing of drug resistance mutations in Mycobacterium tuberculosis strains. Journal of Clinical Microbiology. 2012 December; 50(12):3831-7.
Daum L T, Worthy S A, Yim K C, Nogueras M, Schuman R F, Choi Y W, Fischer G W. A clinical specimen collection and transport medium for molecular diagnostic and genomic applications. Epidemiology and Infection. 2011 November; 139 (11):1764-73.
Daum L T, Canas L C, Arulanandam B P, Niemeyer D, Valdes J J, Chambers J P. Real-time RT-PCR assays for type and subtype detection of influenza A and B viruses. 2007: Influenza and Other Respiratory Viruses 1(4), 167-175.
Chen N, Zhou M, Dong X, Qu J, Gong F, Han Y, Qiu Y, Wang J, Liu Y, Wei Y, Xia J, Yu T, Zhang X, Zhang L. Epidemiological and clinical characteristics of 99 cases of 2019 novel coronavirus pneumonia in Wuhan, China: a descriptive study. Lancet. 2020 Feb. 15; 395(10223):507-513.
Zhu N, Zhang D, Wang W, Li X, Yang B, Song J, Zhao X, Huang B, Shi W, Lu R, Niu P, Zhan F, Ma X, Wang D, Xu W, Wu G, Gao G F, Tan W; China Novel Coronavirus Investigating and Research Team. A Novel Coronavirus from Patients with Pneumonia in China, 2019. N Engl J Med. 2020 Feb. 20; 382(8):727-733.
Araf Y, Akter F, Tang Y D, Fatemi R, Parvez M S A, Zheng C, Hossain M G. Omicron variant of SARS-COV-2: Genomics, transmissibility, and responses to current COVID-19 vaccines. J Med Virol. 2022 May; 94(5):1825-1832.
Forchette L, Sebastian W, Liu T. A Comprehensive Review of COVID-19 Virology, Vaccines, Variants, and Therapeutics. Curr Med Sci. 2021 December; 41(6):1037-1051.
COVID-19 Coronavirus Pandemic. Worldometer website. Available at: https://www.worldometers.info/coronavirus/Accessed: Feb. 17, 2023.
Ritchey M D, Rosenblum H G, Del Guercio K, Humbard M, Santos S, Hall J, Chaitram J, Salerno R M. COVID-19 Self-Test Data: Challenges and Opportunities-United States, Oct. 31, 2021-Jun. 11, 2022. MMWR Morb Mortal Wkly Rep. 2022 Aug. 12; 71(32):1005-1010.
Nagura-Ikeda M, Imai K, Tabata S, Miyoshi K, Murahara N, Mizuno T, Horiuchi M, Kato K, Imoto Y, Iwata M, Mimura S, Ito T, Tamura K, Kato Y. Clinical Evaluation of Self-Collected Saliva by Quantitative Reverse Transcription-PCR (RT-qPCR), Direct RT-qPCR, Reverse Transcription-Loop-Mediated Isothermal Amplification, and a Rapid Antigen Test To Diagnose COVID-19. J Clin Microbiol. 2020 Aug. 24; 58(9).
Ferté T, Ramel V, Cazanave C, Lafon M E, Bébéar C, Malvy D, Georges-Walryck A, Dehail P. Accuracy of COVID-19 rapid antigenic tests compared to RT-PCR in a student population: The StudyCov study. J Clin Virol. 2021 August; 141:104878.
LumiraDx Fast Lab Solutions. SARS-COV-2 RNA Star Complete. Instructions for Use. Available at: https://www.lumiradx.com/assets/pdfs/fast-lab-solutions/sd-com-art-00071-r.4-lumiradx-sars-cov-2-rna-star-complete-quick-reference-instructions-eua_fdafinal.pdf?v=1
Copan Diagnostics. Copan Universal Transport Medium (UTM). Instructions for Use. Available at: https://nvrl.ucd.ie/sites/default/files/uploads/pdfs/UTM-RT_Flocked_Polyester_Swabs.pdf
Becton Dickinson. BD Universal Transport Medium. Instructions for Use. Available at: https://www.bd.com/resource.aspx?IDX=14053
Ertell. K. Pacific Northwest National Laboratory. A Review of Toxicity and Use and Handling Considerations for Guanidine, Guanidine Hydrochloride, and Urea. Available at: www.pnnl.gov/main/publications/external/technical_reports/PNNL-15747.pdf
Centers for Disease Control and Prevention (CDC). Lab Alert: Important Update about Molecular Transport Media (MTM) and Cyanide Gas Available at: www.cdc.gov/locs/2020/important_update_about_mtm_and_cyanide_gas.html
X, Wang L, Sakthivel S K, Whitaker B, Murray J, Kamili S, Lynch B, Malapati L, Burke S A, Harcourt J, Tamin A, Thornburg N J, Villanueva J M, Lindstrom S. US CDC Real-Time Reverse Transcription PCR Panel for Detection of Severe Acute Respiratory Syndrome Coronavirus 2. Emerg Infect Dis. 2020 August; 26(8):1654-65.
Qiagan QIAamp RNA Viral Mini Handbook. July 2020. Available at: www.qiagen.com/dk/resources/resourcedetail?id=c80685c0-4103-49ea-aa72-8989420e3018&lang=en.
LumiraDx SARS-COV-2 RNA Star Complete. LumiraDx (2022). Available at: https://www.lumiradx.com/assets/pdfs/fast-lab-solutions/sars-cov-2-rna-star-ifu-for-ruo-ous.pdf?v=1
MEDCALC Statistical Software. Diagnostic Test Evaluator Software. Available at: www.medcalc.org/calc/diagnostic_test.php
La Scola B, Le Bideau M, Andreani J, Hoang V T, Grimaldier C, Colson P, Gautret P, Raoult D. Viral RNA load as determined by cell culture as a management tool for discharge of SARS-COV-2 patients from infectious disease wards. Eur J Clin Microbiol Infect Dis. 2020 June; 39(6):1059-1061.

### REFERENCED PATENT APPLICATION PUBLICATIONS

US20120100529—Fischer and Daum. Biological specimen collection and transport system and methods of use.
US20120088231—Fischer and Daum, Biological specimen collection/transport compositions and methods.
US20110281754—Fischer and Daum, Compositions and methods for detecting, identifying and quantitating mycobacterial-specific nucleic acids.
US20130040288—Fischer and Daum, Biological specimen collection and transport system and method of use.

We claim:

1. A method for rapid inactivation of nucleases (RNase and DNase) in biological matrices where preservation and stabilization of RNA and DNA are critical to downstream nucleic acid detection and characterization processes, the method comprising the steps of:
preparing an aptamer binding buffer solution;
collecting a sample comprising biological matrices;
combining and mixing the sample and the aptamer binding buffer solution into a first mixture;
allowing RNase and DNase to bind to aptamer in the first mixture;
displacing and segregating the aptamer bound RNase and DNase to a first location in the first mixture;
withdrawing a quantity of residual unbound aqueous material from a second location in the first mixture, the second location separated from the first location, the quantity of residual unbound aqueous material comprising preserved and stabilized RNA and DNA material.

2. A method for utilization of anti-nuclease aptamers in sample/specimen collection medium for inactivation of nucleases (RNase and DNase) from collected biological matrices where preservation and stabilization of RNA and DNA are critical to downstream nucleic acids detection and characterization processes, the method comprising the steps of:
preparing a collection medium comprised of reagents that include at least an aptamer and a membrane lysis detergent;
collecting a biological sample fluid or swab comprising biological matrices;
adding, combining and mixing the biological sample and the collection medium into a closed collection container to form a collection mixture;
performing at least partial lysis of the collected biological sample to release nucleic acids and proteins from cells in the biological matrices;
allowing the nucleases (RNase and DNase) to bind to the aptamer in the collection mixture; and
inactivating the nucleases to enhance stability and preservation of RNA and DNA from the biological sample;
whereby the method provides a multi-use collection system suitable for testing and characterizing nucleic acids, proteins and antigens with or without the use of prefatory nucleic acid extraction or other purification steps.

3. A formulation for a sample collection medium containing an aptamer or collection of aptamers that bind with high affinity and subsequently inactivate nucleases in a collected sample:
the collection medium contains at least one buffer, at least one sugar, at least one salt, at least one detergent, at least one chelating agent, and at least one PCR enhancer, the combined collection medium buffered between 7 and 7.4 with HCl;
the medium contains TRIS, sucrose, sodium acetate, Tween-20, EDTA, betaine, and glycerol, it includes, but is not limited to, the aptamers defined by SEQ ID NO: 1-SEQ ID NO: 5 described herein for RNase inactivation;
the aptamers inactivate RNase at 8,800-fold to 10,200-fold and preserve collected samples at ambient and elevated ambient temperatures up to 30° C.;
the collection medium containing a collected sample can be used with nucleic acid extraction or used directly without extraction in molecular tests, including PCR;
the collection medium containing collected samples can be utilized for multiple diagnostic, detection, and characterization methods for molecular nucleic acid tests and genomic sequencing applications, ELISA, protein, lateral flow, and rapid antigen tests;
wherein the collection medium containing collected sample can be utilized for multiple diagnostic, detection, and characterization methods molecular nucleic acids tests and genomic sequencing applications, ELISA, protein, lateral flow, and rapid antigen tests.
